(19) United States
(12) Patent Application Publication
(10) Pub. No.: US 2022/0280107 A1
(43) Pub. Date: Sep. 8, 2022

(54) ANALYSIS SYSTEM AND ANALYSIS METHOD

(71) Applicants: Murata Manufacturing Co., Ltd., Nagaokakyo-shi (JP); The University of Electro-Communications, Tokyo (JP)

(72) Inventors: Toru SHIMUTA, Nagaokakyo-shi (JP); Keiki TAKADAMA, Chofu-shi (JP); Ryo TAKANO, Chofu-shi (JP)

(21) Appl. No.: 17/750,847

(22) Filed: May 23, 2022

Related U.S. Application Data
(63) Continuation of application No. PCT/JP2020/031470, filed on Aug. 20, 2020.

(30) Foreign Application Priority Data
Nov. 25, 2019 (JP) 2019-212476

(51) Int. Cl. A61B 5/00; A61B 5/0205; A61B 5/024

(52) U.S. Cl. CPC A61B 5/4815; A61B 5/0205; A61B 5/4035; A61B 5/486; A61B 5/6822; A61B 5/02438; A61B 5/4857; A61B 5/742

(57) ABSTRACT

An analysis system and method are provided for analyzing physical information and acquiring biological data of a user from a biological data acquisition sensor. The exemplary analysis system includes a circadian rhythm calculator that calculates an average circadian rhythm of the user; an autonomic nerve analyzer that performs autonomic nerve analysis based on a variation in the biological data; and a physical information analyzer that weights an autonomic nerve analysis result analyzed by the autonomic nerve analyzer with a weighting coefficient set based on a measurement time at which the biological data of the user is acquired and a cycle of the average circadian rhythm. The physical information analyzer also estimates a change in a circadian rhythm with respect to the average circadian rhythm based on the weighted autonomic nerve analysis result.

### CROSS REFERENCE TO RELATED APPLICATIONS

This application is a continuation of PCT Application No. PCT/JP2020/031470, filed Aug. 20, 2020, which claims priority to Japanese Patent Application No. 2019-212476, filed Nov. 25, 2019, the entire contents of each of which are hereby incorporated in their entirety.

### FIELD OF THE INVENTION

The present invention relates to an analysis system and an analysis method for analyzing physical information.

### BACKGROUND

JP 4421507 B2 discloses a sleepiness prediction device considering daytime activity, time, and a sleep state. The sleepiness prediction device described in JP 4421507 B2 measures a sleep state related value related to a sleep state of a subject, and measures a daytime activity related value related to a daytime activity of the subject. The sleepiness prediction device described in JP 4421507 B2 calculates the accumulated sleepiness level predicted to be accumulated by the sleep history and the daytime activity of the subject based on the sleep state related value and the daytime activity related value, and ALSO calculates the biological rhythm sleepiness level based on the biological rhythm changing with time. Accordingly, the sleepiness prediction device described in JP 4421507 B2 calculates a total sleepiness level corresponding to time based on the accumulated sleepiness level and the biological rhythm sleepiness level.

### SUMMARY OF THE INVENTION

In recent years, there has been a demand for an analysis system and an analysis method configured for analyzing physical information. Thus, in an exemplary aspect, an analysis system is provided for analyzing physical information and includes a biological data acquisition sensor that acquires biological data of a user; a circadian rhythm calculator that calculates an average circadian rhythm of the user; an autonomic nerve analyzer that performs autonomic nerve analysis based on a variation in the biological data; a weighting coefficient calculator that calculates a weighting coefficient for weighting an autonomic nerve analysis result analyzed by the autonomic nerve analyzer based on a measurement time at which biological data of the user is measured and a cycle of the average circadian rhythm; and a physical information analyzer that weights the autonomic nerve analysis result by the weighting coefficient calculated by the weighting coefficient calculator, and estimates a change in a circadian rhythm with respect to the average circadian rhythm based on the weighted autonomic nerve analysis result.

Moreover, in another exemplary aspect, an analysis method is provided for analyzing physical information by a computer and includes: acquiring biological data of a user; performing autonomic nerve analysis based on a variation in the biological data of the user; calculating an average circadian rhythm of the user; calculating a weighting coefficient for weighting an autonomic nerve analysis result based on a measurement time at which the biological data of the user is measured and a cycle of the average circadian rhythm; weighing the autonomic nerve analysis result by the calculated weighting coefficient; and estimating a change in a circadian rhythm with respect to the average circadian rhythm based on the weighted autonomic nerve analysis result.

According to the exemplary system and method of the present invention, the physical information can be analyzed.

### BRIEF DESCRIPTION OF THE DRAWINGS

FIG. 1 is a block diagram showing a schematic configuration of an example of an analysis system according to a first exemplary embodiment.

FIG. 2 is a block diagram showing a schematic configuration of a measurement device in the analysis system according to the first exemplary embodiment.

FIG. 3 is a diagram showing an example of an average circadian rhythm.

FIG. 4 is a flowchart showing an example of calculation of the average circadian rhythm in the analysis system according to the first exemplary embodiment.

FIG. 5 is a flowchart showing an example of a method for analyzing physical information according to the first exemplary embodiment.

FIG. 6 is a diagram showing an example of a relationship between a period of the average circadian rhythm and a weighting coefficient.

FIG. 7 is a diagram showing an example of a correlation between an autonomic nerve analysis result, a circadian rhythm change, and a ratio of light sleep.

FIG. 8 is a diagram showing an example of determination of a ratio of light sleep based on the autonomic nerve analysis result and the circadian rhythm change.

FIG. 9 is a diagram showing an example of a correlation between the autonomic nerve analysis result and a time shift of the circadian rhythm.

FIG. 10 is a diagram showing an example of determination of the time shift between the autonomic nerve analysis result and the circadian rhythm.

FIG. 11 is a diagram showing an example of an output displayed by the analysis system according to the first exemplary embodiment.

FIG. 12 is a block diagram showing a schematic configuration of an example of an analysis system according to a second exemplary embodiment.

FIG. 13 is a block diagram showing a schematic configuration of an example of an analysis system according to a third exemplary embodiment.

FIG. 14 is a schematic view of an example of a gripping-type measurement device.

FIG. 15 is a schematic view of an example of a neck-wearing-type measurement device.

FIG. 16 is a schematic view of an example of a wristwatch-type measurement device.

FIG. 17 is a schematic view of an example of a chest-attaching-type measurement device.

### DETAILED DESCRIPTION OF THE EMBODIMENTS

In recent years, a circadian rhythm has been analyzed as a method for analyzing physical information of a user. The circadian rhythm is a 24 hour cycle rhythm of an organism, and examples of the circadian rhythm include variations in blood pressure, body temperature, heart rate, and hormone secretion in one day. The circadian rhythm is considered to be correlated with the autonomic nervous system and sleep.

As described above, JP 4421507 B2 discloses a sleepiness prediction device considering daytime activity, time, and a sleep state. The sleepiness prediction device described in JP 4421507 B2 measures the sleep state related value and the daytime activity related value, and calculates the accumulated sleepiness level. However, in the sleepiness prediction device described in JP 4421507 B2, much of the required data is acquired from the user, which is a burden on the user.

The sleepiness prediction device described in JP 4421507 B2 calculates the sleepiness level, but does not disclose analysis of the circadian rhythm, improvement of the quality of sleep by adjusting the circadian rhythm, and the like.

In view of these limitations, an analysis system is provided according to an exemplary aspect for analyzing physical information and includes a biological data acquisition sensor that acquires biological data of a user; a circadian rhythm calculator that calculates an average circadian rhythm of the user; an autonomic nerve analyzer that performs autonomic nerve analysis based on a variation in the biological data; a weighting coefficient calculator that calculates a weighting coefficient for weighting an autonomic nerve analysis result analyzed by the autonomic nerve analyzer based on a measurement time at which biological data of the user is measured and a cycle of the average circadian rhythm; and a physical information analyzer that weights the autonomic nerve analysis result by the weighting coefficient calculated by the weighting coefficient calculator, and estimates a change in a circadian rhythm with respect to the average circadian rhythm based on the weighted autonomic nerve analysis result. With such a configuration, the change in the circadian rhythm can be estimated and analyzes as one of the physical information.

In the analysis system, the biological data may include at least a heart rate or a pulse rate. With such a configuration, the physical information can be analyzed based on the heart rate or the pulse rate.

In the analysis system, the circadian rhythm calculator may calculate the average circadian rhythm based on the biological data acquired by the biological data acquisition sensor. With such a configuration, the average circadian rhythm can be more accurately calculated based on the biological data.

The analysis system may further include an input device that inputs sleep information of the user, and the circadian rhythm calculator may calculate the average circadian rhythm based on the sleep information input by the input device. With such a configuration, the average circadian rhythm can be easily calculated based on the sleep information, and the physical information can be easily analyzed.

When the measurement time is in a range of a cycle of −⅛ or more and ⅜ or less of a maximum value peak of the average circadian rhythm, the weighting coefficient calculator may increase the weighting coefficient as compared with a case where the measurement time is in other ranges. With such a configuration, the estimation accuracy of the circadian rhythm can be improved, and the physical information can be more accurately analyzed.

Moreover, the physical information analyzer may correct the weighted autonomic nerve analysis result when a heart rate or a pulse rate of the user is larger than a predetermined threshold. With such a configuration, the estimation accuracy of the circadian rhythm can be improved, and the physical information can be more accurately analyzed.

The physical information analyzer may estimate a change in the circadian rhythm based on at least one of a time shift of a maximum value peak of the circadian rhythm from the average circadian rhythm, a time shift of a minimum value peak, a decrease in amplitude, and multimodality. With such a configuration, the physical information of the user can be analyzed in more detail.

Moreover, the physical information analyzer may further estimate quality of sleep and activity preference of the user based on the weighted autonomic nerve analysis result. With such a configuration, the physical information of the user can be analyzed in more detail.

The analysis system may further include a presentation unit that presents presentation information including advice for improving the circadian rhythm, and the physical information analyzer may generate the presentation information based on a change in the circadian rhythm. With such a configuration, advice can be presented for improving the circadian rhythm to the user.

The physical information analyzer may calculate a predicted circadian rhythm based on the weighted autonomic nerve analysis result, and the presentation information may include the average circadian rhythm and the predicted circadian rhythm. With such a configuration, the information on the change in the circadian rhythm can be presented to the user.

The analysis system may further include a notification unit that provides notification of a timing at which the biological data is measured. With such a configuration, the biological data can be acquired at an appropriate timing, and the estimation accuracy of the circadian rhythm can be improved.

The biological data acquisition sensor may be built in an attaching type or wearing-type measurement device. With such a configuration, the measurement device can be easily attached to the user, and the biological data can be easily acquired.

The measurement device may be a device attached to or worn on a neck of the user, and may include a temperature adjuster that adjusts a temperature of the neck of the user. With such a configuration, the disturbance of the circadian rhythm can be suppressed.

The analysis system may further include an activity amount measurement device that measures activity amount data of the user, and the physical information analyzer may correct the weighted autonomic nerve analysis result based on the activity amount data measured by the activity amount measurement device. With such a configuration, since the circadian rhythm can be estimated based on the autonomic nerve analysis result with high reliability, the estimation accuracy of the circadian rhythm can be improved.

According to an exemplary aspect, an analysis system is provided for analyzing physical information and includes one or a plurality of measurement devices; one or a plurality of control terminals that communicate with the one or the plurality of measurement devices; and a server that communicates with the one or the plurality of control terminals. In this aspect, the one or the plurality of measurement devices include a biological data acquisition sensor that acquires biological data of a user; and a first communicator that transmits the biological data acquired by the biological data acquisition sensor to the one or the plurality of control terminals. In addition, the one or the plurality of control terminals include a presentation unit that presents presentation information for improving a circadian rhythm; and a second communicator that transmits the biological data to the server and receives the presentation information from the server. Yet further, the server includes a circadian rhythm calculator that calculates an average circadian rhythm of the user; an autonomic nerve analyzer that performs autonomic nerve analysis based on a variation in biological data of the user among the biological data; a weighting coefficient calculator that calculates a weighting coefficient for weighting an autonomic nerve analysis result analyzed by the autonomic nerve analyzer based on a measurement time at which the biological data of the user is measured and a cycle of the average circadian rhythm; a physical information analyzer that weights the autonomic nerve analysis result by the weighting coefficient calculated by the weighting coefficient calculator, estimates a change in the circadian rhythm with respect to the average circadian rhythm based on the weighted autonomic nerve analysis result, and creates the presentation information based on the change in the circadian rhythm; and a third communicator that receives the biological data from the control terminal and transmits the presentation information to the control terminal.

With such a configuration, the change in the circadian rhythm can be estimated and analyzed as one of the physical information.

According to another exemplary aspect, an analysis method is provided for analyzing physical information by a computer and includes acquiring biological data of a user; performing autonomic nerve analysis based on a variation in the biological data of the user; calculating an average circadian rhythm of the user; calculating a weighting coefficient for weighting an autonomic nerve analysis result based on a measurement time at which the biological data of the user is measured and a cycle of the average circadian rhythm; weighting the autonomic nerve analysis result by the calculated weighting coefficient; and estimating a change in a circadian rhythm with respect to the average circadian rhythm based on the weighted autonomic nerve analysis result.

With such a configuration, the change in the circadian rhythm can be estimated and analyzed as one of the physical information.

Hereinafter, exemplary embodiments of the present invention will be described with reference to the accompanying drawings. It is noted that the following description is merely exemplary in nature and is not intended to limit the present disclosure, an object for application, or a usage. Furthermore, the drawings are schematic, and ratios of dimensions and the like do not necessarily match actual ones.

### First Exemplary Embodiment

### [Overall Configuration]

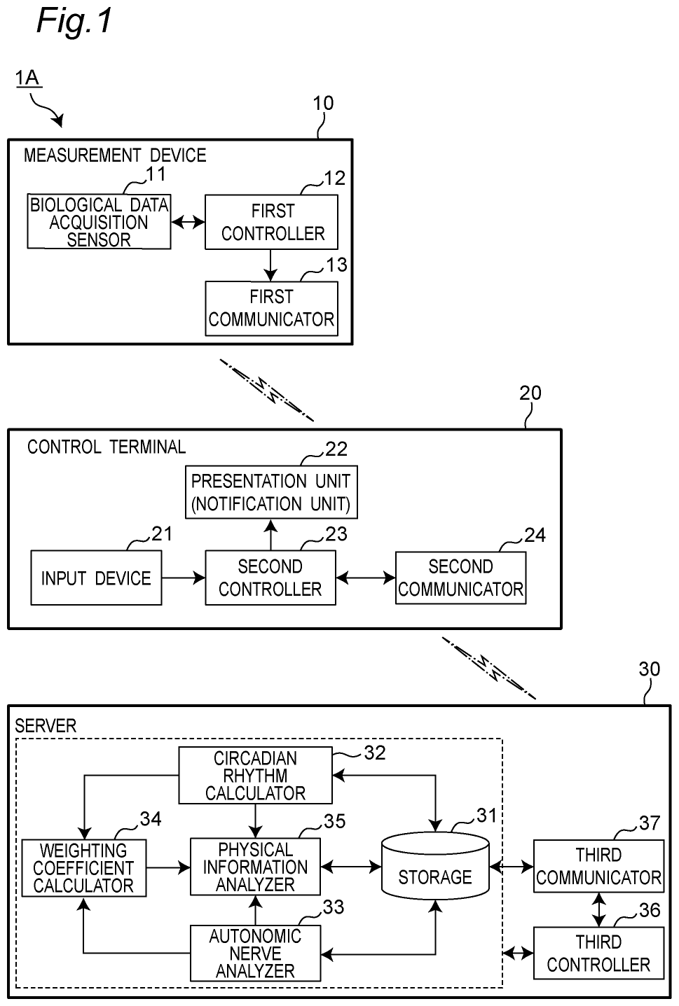

FIG. 1 is a block diagram showing a schematic configuration of an analysis system 1A according to a first exemplary embodiment. As shown in FIG. 1, the analysis system 1A includes a measurement device 10, a control terminal 20, and a server 30. The analysis system 1A is a system that analyzes physical information of the user. In the first embodiment, the analysis system 1A estimates and analyzes a change in a circadian rhythm of the user as physical information.

### <Measurement Device>

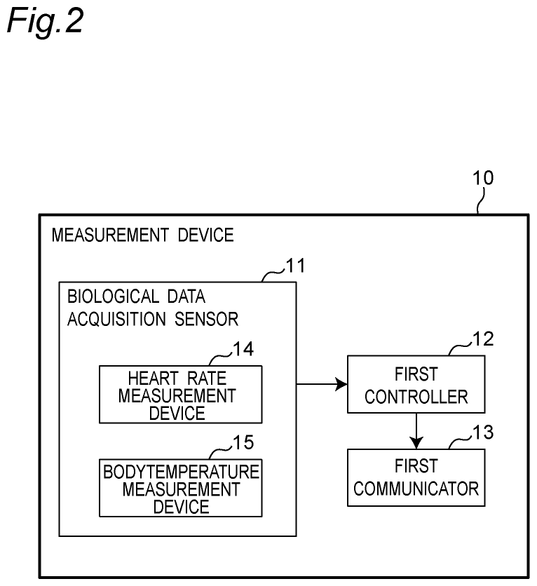

The measurement device 10 is a device that measures biological data of the user. FIG. 2 is a block diagram showing a schematic configuration of the measurement device 10 in the analysis system 1A according to the first embodiment. As shown in FIGS. 1 and 2, the measurement device 10 includes a biological data acquisition sensor 11, a first controller 12, and a first communicator 13.

The biological data acquisition sensor 11 is configured to acquire biological data of the user. The biological data includes, for example, a daily variation in vital information of at least one of body temperature, heart rate, pulse rate, respiration, brain waves, and blood pressure. In the first embodiment, the biological data acquisition sensor 11 acquires biological data including at least a heart rate. Note that the biological data acquisition sensor 11 may acquire biological data including a pulse rate instead of the heart rate in an alternative aspect. The heart rate and the pulse rate of the biological data are easily measured, and the accuracy of analysis of the physical information is improved.

In the first embodiment, the biological data acquisition sensor 11 acquires the biological data a plurality of times when the user is awake. For example, the biological data acquisition sensor 11 acquires the biological data five or more times in one day.

As a measurement condition of the biological data, it is preferable that the user is in a resting state in a sitting position. The resting state means a state of being quiet without moving the body. By measuring the biological data when the user is in the resting state, the estimation accuracy of the circadian rhythm can be improved based on the biological data to be described later. In addition, it is preferable to acquire biological data while avoiding exercise (including walking), eating, bathing, and the like.

As shown in FIG. 2, the biological data acquisition sensor 11 includes a heart rate measurement device 14 and a body temperature measurement device 15. The heart rate measurement device 14 is a heart rate sensor that measures the heart rate of the user. As the heart rate sensor, an electrocardiographic sensor or a ballistocardiologic sensor can be used in exemplary aspects. The body temperature measurement device 15 is a body temperature sensor that measures the body temperature of the user. As the body temperature sensor, a chip thermistor or a resistance temperature detector can be used in exemplary aspects.

In addition, the heart rate during sleep can be estimated by an installation-type body motion sensor. For example, a measurement device 10 including a sheet-type body motion sensor may be installed under a mattress, and body motion data wirelessly output may be received by the control terminal 20 and transmitted to the server 30. The server 30 may analyze the heart rate for autonomic analysis.

In the case of measuring the pulse rate, the biological data acquisition sensor 11 may include a pulse sensor. As the pulse rate sensor, a photoelectric pulse wave sensor, a piezoelectric pulse wave sensor, or an oxygen saturation sensor can be used, for example.

Further, the biological data acquisition sensor 11 acquires time data when the biological data is acquired. The time data includes a measurement time at which the biological data is measured.

The biological data and the time data acquired by the biological data acquisition sensor 11 are transmitted to the first controller 12.

The first controller 12 integrally controls the components of the measurement device 10. The first controller 12 includes, for example, a memory that stores a program, and a processing circuit (not shown) corresponding to a processor, such as a central processing unit (CPU). In the first controller 12, the processor executes the program stored in the memory. In the first embodiment, the first controller 12 controls the biological data acquisition sensor 11 and the first communicator 13.

The first controller 12 is configured to store the biological data and the time data from the biological data acquisition sensor 11 in the memory, and transmits the biological data and the time data to the first communicator 13.

The first communicator 13 transmits the biological data and the time data to the control terminal 20. For example, the first communicator 13 includes a circuit that communicates with the control terminal 20 in conformity with a predetermined communication standard (for example, LAN, Wi-Fi®, Bluetooth®, USB, HDMI®, controller area network (CAN), and serial peripheral interface (SPI)).

### <Control Terminal>

The control terminal 20 communicates with the measurement device 10 and the server 30. In the first embodiment, the control terminal 20 functions as a repeater that relays the measurement device 10 and the server 30, and controls the measurement device 10. Moreover, the control terminal 20 is a smartphone according to an exemplary aspect.

The control terminal 20 transmits the data acquired by the control terminal 20 to the server 30. The control terminal 20 receives the physical information of the user analyzed by the server 30 from the server 30 and displays the physical information. The control terminal 20 includes an input device 21, a presentation unit 22, a second controller 23, and a second communicator 24.

The input device 21 is a device that receives an input from the user. User information is input to the input device 21. In the first embodiment, sleep information of the user is input in the input device 21. The sleep information includes information on a bedtime and a wake-up time of the user, which may be a time of the current day or the previous day, or may be an average time of several days. The bedtime and the wake-up time may be standard times recognized by the user. The bedtime and the wake-up time of the user are used for simple calculation of an average circadian rhythm to be described later.

Furthermore, a comment from the user may be input to the input device 21. For example, by the user inputting a comment regarding his/her own behavior into the input device 21, information regarding the user's behavior can be recorded. The input device 21 may have a selective button. The input can be simplified by associating the comment with the selective button. As a result, the user's operation can be simplified to reduce botheration of the user. Example activities of those that are likely to affect autonomic nerve activity and body temperature include walking, exercise, eating, bathing, and sleeping, and further include going out, work, a feeling of temperature, a feeling of mood and fatigue, and drowsiness. The comment input can be simplified by associating the comment with the selective button. Furthermore, the user may freely input a comment to the input device 21. As a result, it is also possible to input a comment that does not correspond to the selective button. The measurement conditions can be limited by inputting these contents before and after the measurement of the biological data. It is possible to improve the estimation accuracy by using the contents of the comment as the measurement conditions when the autonomic nerve analysis is performed.

In general, it is know that the autonomic nerve function is affected by gender and age. Moreover, the autonomic nerve function decreases more significantly as the user becomes older. That is, the total power decreases as the user becomes older. Therefore, in order to correct the autonomic nerve analysis result by the gender and the age, information of the gender and the age of the user may be input to the input device 21.

The information input by the input device 21 is transmitted to the second controller 23.

The presentation unit 22 is a device that presents presentation information. The presentation information includes physical information (for example, change in circadian rhythm and/or autonomic nerve analysis result) of the user analyzed by the server 30 and/or an improvement advice based on the physical information analysis result. The presentation unit 22 presents the presentation information by screen display, voice, and/or vibration. The presentation unit 22 includes, for example, a display, a speaker, and/or a vibrator.

In the first embodiment, the presentation unit 22 also functions as a notification unit that provides notification of the acquisition timing of the biological data by the measurement device 10. The notification unit provides notification of the timing of acquiring the biological data when the user is awake. For example, the notification unit provides notification of the timing of acquiring the biological data when the user is in a resting state. Moreover, the notification unit may notify the user of the timing of acquiring the biological data after presenting the presentation information instructing the user to enter the resting state. Furthermore, the notification unit may provide notification of a message for confirming whether or not the user is in a resting state before acquiring the biological data. In this case, the notification unit provides notification of the timing of acquiring the biological data after confirming the input from the user to the input device 21. As a result, the biological data can be acquired at a timing suitable for measurement of the user.

The second controller 23 integrally controls the components of the control terminal 20. In an exemplary aspect, the second controller 23 includes a memory that stores a program, and a processing circuit (not shown) corresponding to a processor such as a central processing unit (CPU). In the second controller 23, the processor executes the program stored in the memory. In the first embodiment, the second controller 23 controls the input device 21, the presentation unit 22, and the second communicator 24.

The second communicator 24 communicates with the measurement device 10 and the server 30. For example, the second communicator 24 includes a circuit that communicates with the server 30 in conformity with a predetermined communication standard (for example, LAN, Wi-Fi®, Bluetooth®, USB, HDMI®, controller area network (CAN), and serial peripheral interface (SPI)).

The second communicator 24 receives the biological data and the time data transmitted from the measurement device 10. The second communicator 24 transmits the biological data and the time data to the server 30. In addition, the second communicator 24 transmits information such as the sleep information input by the input device 21 to the server 30.

### <Server>

The server 30 analyzes the physical information of the user based on the biological data and the time data received from the control terminal 20, and transmits an analysis result to the control terminal 20. The server 30 includes a storage 31, a circadian rhythm calculator 32, an autonomic nerve analyzer 33, a weighting coefficient calculator 34, a physical information analyzer 35, a third controller 36, and a third communicator 37.

The storage 31 stores the biological data, the time data, the sleep information, and the like received by the third communicator 37. In addition, the storage 31 stores the physical information (circadian rhythm, autonomic nerve analysis result, and the like) analyzed by the physical information analyzer 35. The storage 31 can be realized by, for example, a hard disk (HDD), SSD, RAM, DRAM, a ferroelectric memory, a flash memory, a magnetic disk, or a combination thereof.

The circadian rhythm calculator 32 calculates the average circadian rhythm of the user based on the biological data and the time data acquired by the biological data acquisition sensor 11. The average circadian rhythm means an average circadian rhythm of the user, and is different for each user.

It is noted that there is a case where a calculation error is large in the calculation of the circadian rhythm, and the calculation error is also affected by a change in the user's bedtime, wake-up time, or daytime behavior. In addition, the circadian rhythm may change between weekdays and holidays. In order to improve the accuracy of the calculation of the average circadian rhythm, the average circadian rhythm is preferably calculated based on biological data for several days, preferably one week or more. The calculation of the average circadian rhythm will be described later.

In the first embodiment, the circadian rhythm calculator 32 calculates the average circadian rhythm based on the sleep information until biological data for one week or more is accumulated. When the biological data for one week or more is accumulated, the circadian rhythm calculator 32 replaces the average circadian rhythm calculated based on the sleep information with the average circadian rhythm calculated based on the biological data. It is noted that in the first exemplary embodiment, an example has been described in which the circadian rhythm calculator 32 replaces the average circadian rhythm calculated based on the sleep information with the average circadian rhythm calculated based on the biological data, but the present invention is not limited thereto. For example, the circadian rhythm calculator 32 may use the average circadian rhythm calculated based on the sleep information as it is without replacing the average circadian rhythm calculated based on the sleep information with the average circadian rhythm calculated based on the biological data. Alternatively, the circadian rhythm calculator 32 may correct the average circadian rhythm calculated based on the sleep information by using the average circadian rhythm calculated based on the biological data.

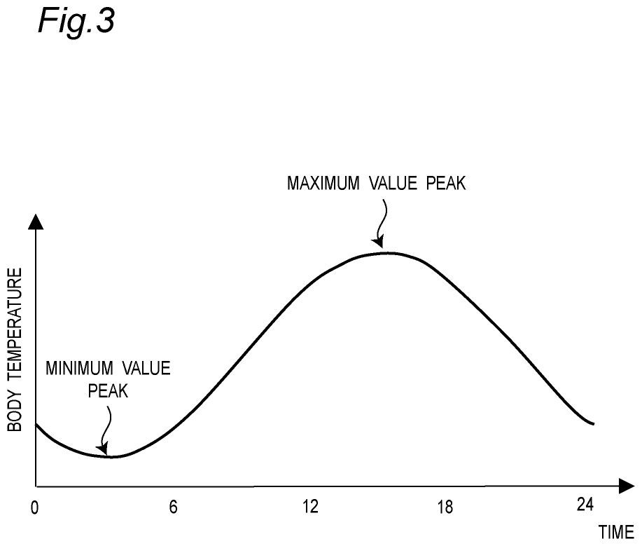

FIG. 3 is a diagram showing an example of the average circadian rhythm. In FIG. 3, the horizontal axis represents time and the vertical axis represents body temperature. FIG. 3 shows an example of the average circadian rhythm based on the body temperature of the user as the biological data. As shown in FIG. 3, the exemplary average circadian rhythm based on the body temperature has a maximum value peak and a minimum value peak, and periodically varies.

The average circadian rhythm may be classified for each type. It is noted that if the classification is too fine, the influence of the error becomes strong, and the correlation may be rather lost. Therefore, about four to eight is appropriate as the number of classifications. For example, the average circadian rhythm can be classified by the cycle and/or peak time of the body temperature variation. The cycle means an interval between local minimum value peaks of the body temperature variation or an interval between maximum value peaks of the body temperature variation.

As the classification of the circadian rhythm, for example, the circadian rhythm can be classified into a morning type (e.g., maximum value peak time: around 16:00, minimum value peak time: around 4:00), an night type (e.g., maximum value peak time: around 22:00, minimum value peak time: around 10:00), an inverted morning type (e.g., maximum value peak time: around 4:00, minimum value peak time: around 16:00), and an inverted night type (e.g., maximum value peak time: around 10:00, minimum value peak time: around 22:00) as a classification based on a difference between a maximum value peak and a minimum value peak of the body temperature. In addition, the circadian rhythm can be classified into a constant type (around 24 hours), a short cycle type (around 20 hours), a long cycle type (around 28 hours), an unknown type (a clear cycle cannot be confirmed), and the like as a classification based on a difference in cycle. Note that the values of the peak time and the cycle time are examples, and the numerical values may be different. In addition, the circadian rhythm usually includes one maximum value peak and one minimum value peak in one day. However, in the circadian rhythm, multimodality including two or more local maximum value peaks and/or local minimum value peaks in one day may occur. The multimodality is also regarded as a kind of disturbance of the circadian rhythm.

It is also noted that in the first exemplary embodiment, the circadian rhythm has been described as a variation of the body temperature, but the circadian rhythm is not limited thereto. The circadian rhythm is calculated by a variation of the biological data. For example, the circadian rhythm may be calculated by a heart rate, a pulse rate, or the like.

The autonomic nerve analyzer 33 performs the autonomic nerve analysis based on the variation of the biological data. In the first embodiment, the autonomic nerve analyzer 33 performs the autonomic nerve analysis based on the variation of the heart rate of the user in the biological data. Specifically, the autonomic nerve analyzer 33 calculates the autonomic nerve activity index (LF, HF, LF/HF, TP, ccvTP) based on the variation of the heart rate when the user is awake. LF is a low-frequency component. HF is a high-frequency component. LF/HF is (ratio of low frequency component/high frequency component). TP is total power ((autonomic nerve activity amount)=LF+HF). ccvTP is a value obtained by correcting TP with the heart rate during the measurement time. The autonomic nerve analyzer 33 calculates at least one of LF, HF, LF/HF, TP, and ccvTP as the autonomic nerve activity index.

The weighting coefficient calculator 34 calculates a weighting coefficient K for weighting the autonomic nerve analysis result analyzed by the autonomic nerve analyzer 33 based on the measurement time at which the biological data of the user is measured and the cycle of the average circadian rhythm. In the first embodiment, the weighting coefficient calculator 34 calculates the weighting coefficient K based on the measurement time at which the heart rate of the user is measured and the cycle of the average circadian rhythm. The weighting coefficient K is calculated based on the measurement time of the heart rate and the cycle of the maximum value peak of the average circadian rhythm. The weighting coefficient K is calculated so as to be larger when the measurement time of the heart rate is in a predetermined time range including the maximum value peak of the average circadian rhythm than when the measurement time is in the other time ranges. The autonomic nerve analysis result can be weighted by the weighting coefficient K. The weighting means adjusting the reliability of the autonomic nerve analysis result. The value of the weighting coefficient K increases as the reliability is higher, and decreases as the reliability is lower.

For example, the maximum value peak time of the circadian rhythm can be an index for determining the quality of sleep of the user. Therefore, the weighting coefficient calculator 34 makes the weighting coefficient near the maximum value peak time of the circadian rhythm larger than the weighting coefficients at other times. For example, the weighting coefficient K near the maximum value peak time of the circadian rhythm may be set to “1”, and the weighting coefficients K at other times may be set to “0”.

The physical information analyzer 35 weights the autonomic nerve analysis result by the weighting coefficient K calculated by the weighting coefficient calculator 34, and estimates the change in the circadian rhythm with respect to the average circadian rhythm based on the weighted autonomic nerve analysis result. As a result, the physical information analyzer 35 analyzes disturbance of the circadian rhythm as one of the physical information.

For example, a case where LF/HF is used as the autonomic nerve activity index will be described. The LF/HF weighted by the weighting coefficient K is referred to as corrected LF/HF. When the corrected LF/HF increases, the disturbance of the circadian rhythm often occurs on the current day or after several days later (e.g., one to three days later). For example, when the minimum value peak time of the average circadian rhythm is around 4:00, the minimum value peak time of the circadian rhythm on the current day or after several days later (e.g., one to three days later) is delayed to around 7:00. In addition, as the corrected LF/HF increases, the amplitude tends to decrease.

It is noted that the above description of the case using LF/HF is an example, and it is possible to estimate the disturbance of the circadian rhythm by using any autonomic nerve activity index among LF, HF, LF/HF, TP, and ccvTP. For example, TP or ccvTP weighted by the weighting coefficient K is referred to as corrected TP or corrected ccvTP. When the corrected TP or the corrected ccvTP increases, the disturbance of the circadian rhythm on the current day or after several days later (e.g., one to three days later) often occurs. For example, in the circadian rhythm on the current day or after several days later (e.g., one to three days later), a decrease in amplitude and a delay of a minimum value peak time occur.

Examples of the disturbance of the circadian rhythm include a shift of a maximum value peak time, a shift of a minimum value peak time, a decrease in amplitude, and multimodality. These may occur alone, but are often combined. For example, when the disturbance of the circadian rhythm is temporary (e.g., several days or less), a shift of a peak time and a decrease in amplitude are likely to occur at the same time.

As described above, the physical information analyzer 35 can estimate the change in the circadian rhythm on the current day or after several days with respect to the average circadian rhythm based on the autonomic nerve analysis result weighted by the weighting coefficient K. Furthermore, the physical information analyzer 35 estimates a shift of a maximum value peak time, a shift of a minimum value peak time, a decrease in amplitude, and multimodality as the change in the circadian rhythm.

The physical information analyzer 35 creates presentation information for improving the circadian rhythm based on the change in the estimated circadian rhythm. For example, the physical information analyzer 35 calculates the predicted circadian rhythm based on the weighted autonomic nerve analysis result. The predicted circadian rhythm means a circadian rhythm after several hours or several days, and indicates prediction of how much the circadian rhythm changes with respect to the average circadian rhythm.

In the first embodiment, the presentation information includes an average circadian rhythm and a predicted circadian rhythm. The presentation information is stored in the storage 31 and transmitted to the control terminal 20 via the third communicator 37. The control terminal 20 presents the presentation information to the presentation unit 22. As a result, the user can know the change (disturbance) of the circadian rhythm with respect to the average circadian rhythm by viewing the presentation information of the presentation unit 22.

Furthermore, the physical information analyzer 35 may create presentation information including improvement advice for adjusting the circadian rhythm in an exemplary aspect. Examples of the improvement advice include proposals such as breathing methods, stretching, yoga, aromatherapy, acupuncture (an attaching type such as circular acupuncture), exercise, and walking.

The physical information (e.g., circadian rhythm, autonomic nerve analysis result, and the like) and the presented information analyzed by the physical information analyzer 35 are stored in the storage 31.

Furthermore, the physical information analyzer 35 can also estimate a REM sleep cycle, a sleep depth, a bedtime, a wake-up time, a sleep time, and the like based on the change in circadian rhythm, and analyze the quality of sleep.

The third controller 36 integrally controls the components of the server 30. The third controller 36 includes, for example, a memory that stores a program, and a processing circuit (not shown) corresponding to a processor such as a central processing unit (CPU). In the third controller 36, the processor executes the program stored in the memory. In the first embodiment, the third controller 36 controls the storage 31, the circadian rhythm calculator 32, the autonomic nerve analyzer 33, the weighting coefficient calculator 34, the physical information analyzer 35, and the third communicator 37.

The third communicator 37 communicates with the control terminal 20. For example, the third communicator 37 includes a circuit that communicates with the control terminal 20 in conformity with a predetermined communication standard (for example, LAN, Wi-Fi®, Bluetooth®, USB, HDMI®, controller area network (CAN), and serial peripheral interface (SPI)).

The third communicator 37 receives the biological data, the time data, and the sleep information transmitted from the control terminal 20. The third communicator 37 transmits the physical information and the presentation information to the control terminal 20.

### [Operation]

An example of an operation (analysis method) of the analysis system 1A will be described. The analysis system 1A calculates the average circadian rhythm in the server 30 based on the biological data measured by the measurement device 10 or the sleep information input to the control terminal 20. The analysis system 1A performs the autonomic nerve analysis based on the heart rate in the biological data, and weights the autonomic nerve analysis result by the weighting coefficient K. The analysis system 1A estimates and analyzes a change in the circadian rhythm with respect to the average circadian rhythm based on the weighted autonomic nerve analysis result.

### <Calculation of Average Circadian Rhythm>

An example of calculation of the average circadian rhythm in the analysis system 1A will be described.

The circadian rhythm calculator 32 calculates a first circadian rhythm based on the sleep information (e.g., bedtime and wake-up time) of the user until biological data for one week or more is acquired, and uses the first circadian rhythm as the average circadian rhythm. When the biological data for one week or more is acquired, the circadian rhythm calculator 32 calculates a second circadian rhythm based on the biological data for one week or more, and uses the second circadian rhythm as the average circadian rhythm. As described above, until the biological data is sufficiently accumulated, the first circadian rhythm simply calculated based on the sleep information of the user is set as the average circadian rhythm. When the biological data is sufficiently accumulated, the second circadian rhythm calculated based on the biological data is set as the average circadian rhythm.

In the first embodiment, the sleep information used for the simple calculation of the first circadian rhythm is information of the bedtime and the wake-up time on the current day or the previous day. The sleep information used for the simple calculation of the first circadian rhythm is not limited to the bedtime and the wake-up time on the current day or the previous day. For example, the sleep information used for the simple calculation of the first circadian rhythm may be information of an average bedtime and an average wake-up time in several days, or may be information of a standard bedtime and wake-up time recognized by the user.

In the first embodiment, the circadian rhythm calculator 32 calculates the average circadian rhythm using the body temperature of the user as the biological data. It is noted that the average circadian rhythm may be calculated using a heart rate, a pulse rate, or the like in addition to the body temperature.

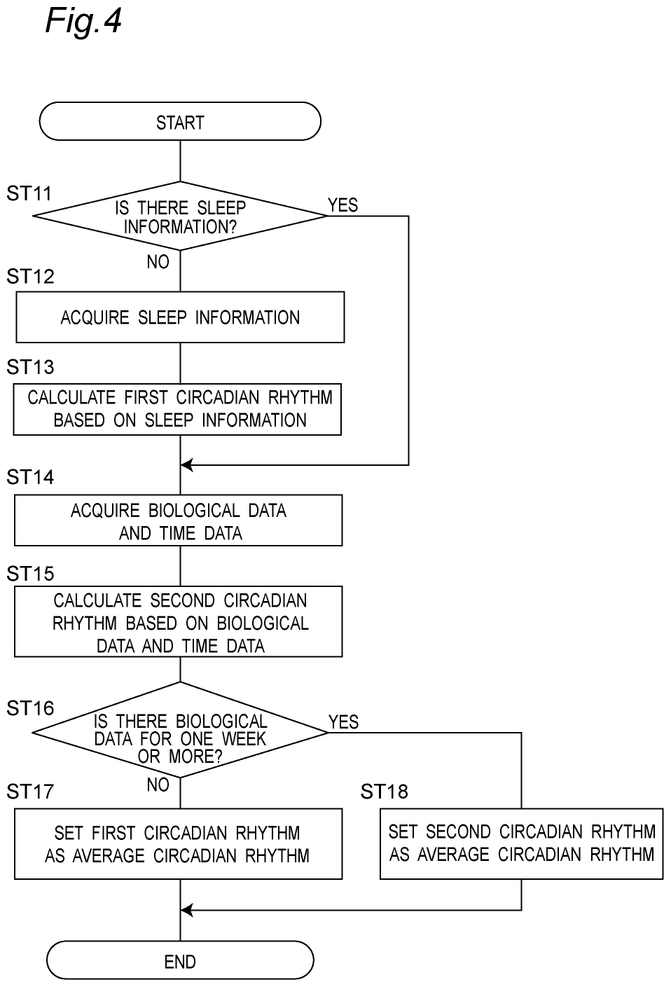

FIG. 4 is a flowchart showing an example of calculation of the average circadian rhythm in the analysis system 1A according to the first embodiment of the present invention.

As shown in FIG. 4, in step ST11, the circadian rhythm calculator 32 determines whether or not there is sleep information of the user. Specifically, the circadian rhythm calculator 32 determines whether or not the sleep information is stored in the storage 31. If there is no sleep information, the flow proceeds to step ST12. If there is sleep information, the flow proceeds to step ST14.

A case where there is no sleep information will be described. In step ST12, the circadian rhythm calculator 32 acquires sleep information. Specifically, the circadian rhythm calculator 32 transmits instruction information to the control terminal 20 via the third communicator 37, and causes the presentation unit 22 of the control terminal 20 to present an instruction for acquiring the sleep information.

The user inputs the sleep information to the input device 21 according to the instruction presented by the presentation unit 22. In the first embodiment, the user inputs the bedtime and the wake-up time to the input device 21. The sleep information input by the input device 21 is transmitted to the server 30 via the second communicator 24 and stored in the storage 31 of the server 30.

As described above, in step ST12, the presentation unit 22 of the control terminal 20 presents the presentation information prompting the user to input the sleep information, and the user is caused to input the sleep information to the input device 21, thereby acquiring the sleep information.

In step ST13, the circadian rhythm calculator 32 calculates the first circadian rhythm based on the sleep information. The first circadian rhythm is a circadian rhythm simply calculated based on the bedtime and the wake-up time of the user.

The circadian rhythm is correlated with the bedtime and the wake-up time of the user. For example, since the bedtime and the wake-up time of the night user are relatively late times, the peak time of the circadian rhythm tends to be delayed. In one example, the circadian rhythm calculator 32 creates in advance a correlation equation or a correlation table indicating a correlation between the bedtime, the wake-up time, and the circadian rhythm, and stores the correlation equation or the correlation table in the storage 31. The circadian rhythm calculator 32 reads the correlation equation or the correlation table from the storage 31, and calculates the first circadian rhythm from the sleep information input by the user and the correlation equation or the correlation table.

Once the sleep information is input by the user, the user may not be required to input the sleep information again. However, after a long period of time (for example, three months or more), the user's lifestyle may change, and the bedtime and the wake-up time may change. Therefore, the input may be obtained periodically (for example, every three months). Alternatively, the user may be requested to input sleep information (e.g., bedtime and wake-up time) a plurality of times. Accordingly, the average bedtime and the average wake-up time may be calculated.

Note that the correlation equation or the correlation table indicating the correlation between the bedtime, the wake-up time, and the circadian rhythm may be created based on the sleep information of a plurality of users. In this case, the sleep information of the plurality of users may be accumulated in the storage 31 of the server 30, and the correlation equation or the correlation table may be created based on the accumulated sleep information.

A case where there is sleep information will be described. In step ST14, the biological data acquisition sensor 11 acquires biological data and time data. Specifically, the circadian rhythm calculator 32 reads the biological data and the time data from the storage 31.

In step ST15, the circadian rhythm calculator 32 calculates the second circadian rhythm based on the biological data and the time data. Specifically, the second circadian rhythm is estimated by organizing the biological data measured when the user is awake at the measurement time, and calculating the cycle of the change in the biological data, the times of the maximum value peak and the minimum value peak of the biological data, and the amplitude of the change in the biological data. In order to improve the estimation accuracy of the circadian rhythm, the number of pieces of biological data acquired by the biological data acquisition sensor 11 in one day is preferably five or more.

Next, the setting of the average circadian rhythm will be described. In step ST16, the circadian rhythm calculator 32 determines whether or not there is biological data for one week or more. Specifically, the circadian rhythm calculator 32 determines whether or not biological data for one week or more is stored in the storage 31. If there is no biological data for one week or more, the flow proceeds to step ST17. If there is biological data for one week or more, the flow proceeds to step ST18.

A case where there is no biological data for one week or more will be described. In step ST17, the circadian rhythm calculator 32 sets the first circadian rhythm as the average circadian rhythm. That is, in a case where biological data for one week or more is not accumulated in the storage 31, the circadian rhythm calculator 32 sets the first circadian rhythm simply calculated based on the sleep information as the average circadian rhythm.

A case where there is biological data for one week or more will be described. In step ST18, the circadian rhythm calculator 32 sets the second circadian rhythm as the average circadian rhythm. That is, in a case where biological data for one week or more is accumulated in the storage 31, the circadian rhythm calculator 32 sets the second circadian rhythm calculated based on the biological data as the average circadian rhythm. As described above, in a case where biological data is not accumulated, the circadian rhythm calculator 32 uses the first circadian rhythm based on the sleep information as the average circadian rhythm. Then, in a case where biological data for one week or more is accumulated, the first circadian rhythm is replaced with the second circadian rhythm, and the second circadian rhythm is used as the average circadian rhythm.

### <Analysis of Physical Information>

Next, an example of analysis of physical information in the analysis system 1A will be described. In the first exemplary embodiment, estimation and analysis of a change in the circadian rhythm will be described as one of physical information.

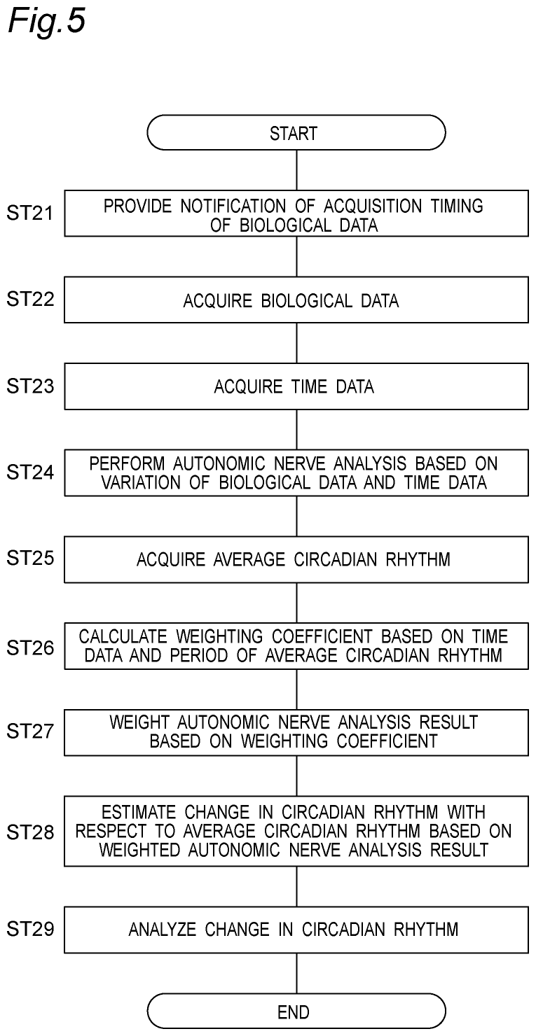

FIG. 5 is a flowchart showing an example of a method for analyzing physical information according to the first embodiment of the present invention.

As shown in FIG. 5, in step ST21, the presentation unit 22 provides notification of the acquisition timing of the biological data. Specifically, the server 30 transmits timing information for acquiring the biological data to the control terminal 20. Upon receiving the information from the server 30, the control terminal 20 presents presentation information instructing the user to acquire the biological data in the presentation unit 22. As a result, the user can be notified of the timing of acquiring the biological data and urge the user to acquire the biological data by the measurement device 10.

Determination criteria of the timing of acquiring the biological data are that the time zone is a time zone in which the degree of importance of the autonomic nerve analysis result is high (for example, the time zone in which the circadian rhythm is in the vicinity of the maximum value peak), and that it can be estimated as the resting state (for example, the activity amount is small, the heart rate or the pulse rate is stable, and the body temperature is stable).

When the user is not in the resting state, the control terminal 20 may prompt the user to be in the resting state by the presentation unit 22 and cause the user to determine whether or not the user is in the resting state. For example, the control terminal 20 may display a message “please rest for 5 minutes” on the presentation unit 22, and may display a message “start measurement if you are in a resting state” 5 minutes after displaying the message.

In step ST22, the biological data acquisition sensor 11 acquires the biological data of the user. In the first embodiment, the biological data acquisition sensor 11 acquires the body temperature and the heart rate of the user as the biological data.

In step ST23, the biological data acquisition sensor 11 acquires time data. Specifically, the biological data acquisition sensor 11 acquires the measurement time when the biological data is acquired.

The biological data and the time data acquired by the biological data acquisition sensor 11 are transmitted to the server 30 via the control terminal 20.

In step ST24, the autonomic nerve analyzer 33 performs the autonomic nerve analysis based on the variation of the biological data of the user. Specifically, the autonomic nerve analyzer 33 calculates the autonomic nerve activity index based on the variation of the heart rate in the biological data acquired in step ST22. The autonomic nerve analyzer 33 calculates at least one of LF, HF, LF/HF, TP, and ccvTP as the autonomic nerve activity index. The autonomic nerve analyzer 33 organizes the calculated autonomic nerve activity indexes by time data. Specifically, the autonomic nerve analyzer 33 organizes the autonomic nerve activity indexes by the measurement time at which the heart rate is measured.

In step ST25, the physical information analyzer 35 acquires an average circadian rhythm. Specifically, the circadian rhythm calculator 32 calculates the average circadian rhythm based on the method shown in FIG. 4. The physical information analyzer 35 acquires the average circadian rhythm calculated by the circadian rhythm calculator 32.

In step ST26, the weighting coefficient calculator 34 calculates the weighting coefficient K based on the time data and the period of the average circadian rhythm. The weighting coefficient calculator 34 calculates the weighting coefficient K for weighting the autonomic nerve analysis result analyzed by the autonomic nerve analyzer 33 based on the measurement time at which the biological data (heart rate) of the user is measured and the cycle of the average circadian rhythm.

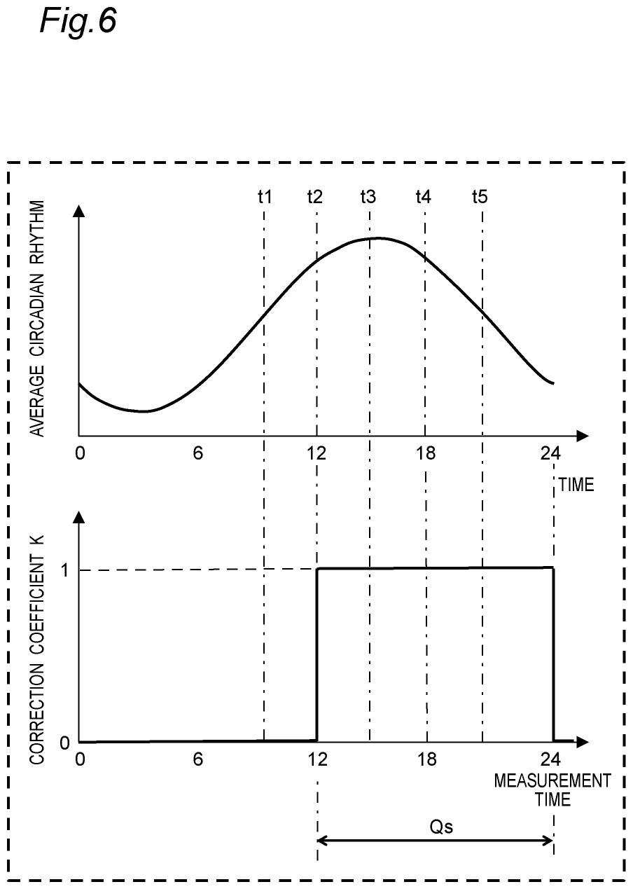

Here, an example of calculation of the weighting coefficient K will be described with reference to FIG. 6. FIG. 6 is a diagram showing an example of a relationship between the period of the average circadian rhythm and the weighting coefficient K. In the example shown in FIG. 6, by adjusting the weighting coefficient K, the reliability of the autonomic nerve analysis result is increased in a case where the measurement time is in a predetermined time range Qs including the maximum value peak of the average circadian rhythm, and the reliability of the autonomic nerve analysis result is lowered in a case where the measurement time is outside the time range Qs. Specifically, when the measurement time is in the predetermined time range Qs including the maximum value peak of the average circadian rhythm, the weighting coefficient K is set to “1”. When the measurement time is outside the predetermined time range Qs including the maximum value peak of the average circadian rhythm, the weighting coefficient K is set to “0”.

The autonomic nerve analysis result based on the biological data measured in the predetermined time range Qs is highly reliable data for determining the quality of sleep of the user. That is, by using the autonomic nerve analysis result in the predetermined time range Qs, the estimation accuracy of the change in the circadian rhythm can be increased. The predetermined time range Qs is preferably set within a range of a cycle of −⅛ or more and ⅜ or less of the maximum value peak of the average circadian rhythm. The “range of a cycle of −⅛ or more and ⅜ or less of the peak of the maximum value” means a range corresponding to −⅛ or more and ⅜ or less with respect to the peak position when one cycle of the graph of the average circadian rhythm is divided into eight equal parts in the time direction. More preferably, the predetermined time range Qs is set in a range of a cycle of ¼ or less of the maximum value peak of the average circadian rhythm.

In the example shown in FIG. 6, biological data is acquired at five timings t1 to t5 between 9:00 and 21:00 in one day. In this example, the timings t1, t2, t3, t4, and t5 indicate around 9:00, around 12:00, around 15:00, around 18:00, and around 21:00, respectively. Furthermore, in the example shown in FIG. 6, the maximum value peak time of the average circadian rhythm is around 15:00. Therefore, the predetermined time range Qs is set to a range from 12:00 to 24:00. In this case, the weighting coefficient calculator 34 sets the weighting coefficient K at the first timing t1 to “0”, and sets the weighting coefficients K at the second to fifth timings t2 to t5 to “1”.

It is noted that the calculation of the weighting coefficient K shown in FIG. 6 is an example, and the calculation of the weighting coefficient K by the weighting coefficient calculator 34 is not limited thereto. For example, the weighting coefficient K may be set to different values in a plurality of time ranges. The weighting coefficient K may be set to increase or decrease in stages based on the maximum value peak time of the average circadian rhythm.

In step ST27, the physical information analyzer 35 weights the autonomic nerve analysis result based on the weighting coefficient K. Specifically, the physical information analyzer 35 multiplies the autonomic nerve activity index calculated by the autonomic nerve analyzer 33 by the weighting coefficient K. In the example of FIG. 6, the autonomic nerve activity index outside the predetermined time range Qs becomes “0”, and the autonomic nerve activity index within predetermined time range Qs remains. In addition, the autonomic nerve analysis result based on the biological data acquired in the resting state is used for estimating a fatigue state. However, in the autonomic nerve analysis result based on the biological data acquired in a state where the sympathetic nerve is enhanced and the heart rate greatly increases, such as exercise or drinking that is not in the resting state, the estimation accuracy of the fatigue state decreases. Therefore, the physical information analyzer 35 may correct the weighted autonomic nerve analysis result when the heart rate of the user is larger than a predetermined threshold. For example, the physical information analyzer 35 may correct the weighted autonomic nerve analysis result when the heart rate is larger than the (average/median) heart rate of the user in normal times by a certain fixed amount (for example, an increase of 20% from the average value).

For example, after the autonomic nerve analysis result is weighted by the weighting coefficient K, the weighted autonomic nerve analysis result is multiplied by the correction coefficient K1. The correction coefficient K1 may be 0.5. The reliability can be lowered by multiplying the weighted autonomic nerve analysis result by the correction coefficient K1.

The correction coefficient K1 may be changed according to an increase rate of the heart rate. For example, in a case where the heart rate increases by 20% or more and less than 40% of the normal time, the correction coefficient K1 may be set to 0.5, and in a case where the heart rate increases by 40 or more and less than 60% of the normal time, the correction coefficient K1 may be set to 0.25.

Alternatively, the correction coefficient K1 may be calculated by the following Expression 1:

(Correction coefficient K1)=(heart rate in normal state)/((heart rate)−(heart rate in normal state))/(constant a)

Here, the constant “a” is set to an arbitrary value. For example, the constant “a” is 5 or more and 20 or less.

In step ST28, the physical information analyzer 35 estimates a change in the circadian rhythm with respect to the average circadian rhythm based on the weighted autonomic nerve analysis result. For example, in a case where the weighted autonomic nerve analysis result exceeds a predetermined threshold Sa, the physical information analyzer 35 predicts that the circadian rhythm is disturbed.

In the first embodiment, the predetermined threshold Sa is determined based on an average value H1 and a standard deviation 6 of the autonomic nerve analysis result before weighting. For example, the predetermined threshold Sa is calculated by the following Expression 2:

(Threshold Sa)=(average H1)+(standard deviation σ)×(constant b)

Here, the constant “b” is set to an arbitrary value. For example, the constant “b” is set to 1.5. Note that the constant “b” is not limited to 1.5, and may be set to another value.

When determining that the weighted autonomic nerve analysis result exceeds the predetermined threshold Sa, the physical information analyzer 35 predicts that the circadian rhythm on the current day or after several days deviates from the average circadian rhythm.

The deviation amount of the circadian rhythm when the predetermined threshold Sa is exceeded is calculated by, for example, the following Expression 3:

(Deviation amount Va)=(constant b)^{c}×(constant d)

“c” is a power of the constant “b”. Each of “c” and “d” is set to an arbitrary value. It is noted that the deviation amount Va is indicated by a deviation of the maximum value peak time of the circadian rhythm.

The physical information analyzer 35 may set a plurality of thresholds Sa. In a case where a first threshold Sa1, a second threshold Sa2, and a third threshold Sa3 are calculated by an expression of Expression 2, the constants “b” are set to different numerical values. Deviation amounts Va1, Va2, and Va3 of the circadian rhythm with respect to the average circadian rhythm in a case where the first threshold Sa1, the second threshold Sa2, and the third threshold Sa3 are exceeded can also be calculated by an expression of Expression 3. A correspondence table of the first threshold Sa1, the second threshold Sa2, and the third threshold Sa3, and the deviation amounts Va1, Va2, and Va3 may be created and stored in the storage 31. In this case, the physical information analyzer 35 can easily calculate the deviation amount of the circadian rhythm with reference to the correspondence table when each threshold is exceeded.

In addition, fatigue accumulates, and fatigue that cannot be recovered by sleep or daytime behavior affects the degree of fatigue on the next day. Therefore, the estimation accuracy of the change in the circadian rhythm can be improved by using data for several days rather than data for one day. That is, in a case where the autonomic nerve analysis result weighted using the data for several days exceeds the threshold, the estimation accuracy of the disturbance of the circadian rhythm is improved as compared with the case of the data for one day. In addition, the degree of the disturbance increases.

Furthermore, the physical information analyzer 35 may also use a change in the biological data on the current day of measurement of the biological data (estimation result up to that point in time of the circadian rhythm) for analysis. As a result, the estimation accuracy of the change in the circadian rhythm can be improved.

In this manner, the physical information analyzer 35 estimates a change (e.g., a disturbance) in the circadian rhythm with respect to the average circadian rhythm based on the weighted autonomic nerve analysis result.

In step ST29, the physical information analyzer 35 analyzes a change in the circadian rhythm. Specifically, the physical information analyzer 35 calculates the predicted circadian rhythm after several hours or several days based on the weighted autonomic nerve analysis result. The physical information analyzer 35 creates presentation information including the average circadian rhythm and the predicted circadian rhythm.

The physical information analyzer 35 transmits the presentation information to the control terminal 20. The control terminal 20 presents the presentation information by the presentation unit 22. As a result, the user can know the change in the circadian rhythm by viewing the presentation information presented in the presentation unit 22.

The physical information analyzer 35 may create presentation information including advice for improving the circadian rhythm based on a change in the circadian rhythm. For example, in a case where the physical information analyzer 35 estimates the disturbance of the circadian rhythm, the deterioration in the quality of sleep, and the decrease in the activity preference, the presentation information including the improvement advice and/or the autonomic nerve analysis result target value may be created.

For example, in a case where the corrected LF/HF is high, the physical information analyzer 35 proposes, as improvement advice, a breathing method, stretching, yoga, aromatherapy, acupuncture (e.g., an attaching type such as circular acupuncture), and the like together with the autonomic nerve analysis result target value. Alternatively, when the corrected TP is high, the physical information analyzer 35 proposes improvement advice such as exercise and walking together with the autonomic nerve analysis result target value. After the user gives the improvement advice, the physical information analyzer 35 can perform the autonomic nerve analysis again to determine whether or not the autonomic nerve analysis result target value has been achieved. When the weighted autonomic nerve analysis result (autonomic nerve activity index such as corrected TP, corrected LF/HF, and the like) is large, the change in the quality of sleep occurs together with the change in the circadian rhythm. Examples of the index of the quality of sleep include a sleep time, a time or a ratio of light sleep (for example, awakening, REM sleep, non-REM sleep stage 1, and the like) in the sleep time, a REM sleep cycle, the number/frequency of awakening from sleep, a difference between the time of going to bed and the time of falling asleep, and a difference between the time of awakening and the wake-up time. Among them, a particularly high correlation with the weighted autonomic nerve analysis result (for example, corrected TP, corrected LF/HF) is a ratio of light sleep to the sleep time. In a case where the weighted autonomic analysis result is large, the ratio of light sleep increases. In a case where the weighted autonomic nerve analysis result (for example, corrected TP, corrected LF/HF) is large, there are a case where the quality of sleep changes after the change in the circadian rhythm occurs (for example, one to three days later), and a case where the change in the circadian rhythm occurs after the change in the quality of sleep.

In addition, if the circadian rhythm is disturbed and the quality of sleep is deteriorated, the performance during the day after the next day is deteriorated. Whether or not it is suitable for exhibiting performance is expressed by activity preference. The activity preference may be calculated from the circadian rhythm up to the previous day, the quality of sleep, and the autonomic nerve analysis result up to the current day (unweighted LF/HF, TP). Factors that decrease the activity preference are disturbance of the circadian rhythm until the previous day, deterioration of the quality of sleep, and deterioration of TP that is not weighted on the current day. Although LF/HF also gives an influence, LF/HF values suitable for exhibiting performance have large individual differences, and thus LF/HF can also be added to a factor of activity preference calculation by accumulating user data.

As described above, the corrected TP is correlated with the quality of sleep after that day. For example, when the corrected TP increases, the quality of sleep tends to decrease. In addition, the autonomic nerve analysis result has a correlation with activity preference. For example, when the TP that is not weighted increases, the activity preference tends to increase. The higher the activity preference, the more suitable the activity. The corrected LF/HF is correlated with disturbance of the circadian rhythm after that day. For example, when the corrected LF/HF is high, the circadian rhythm is easily disturbed.

Using the correlation as described above, the physical information analyzer 35 can analyze the change in the circadian rhythm and calculate physical information such as the quality of sleep and the activity preference.

The analysis system 1A is configured to estimate and analyze the change in the circadian rhythm by performing steps ST21 to ST29 described above.

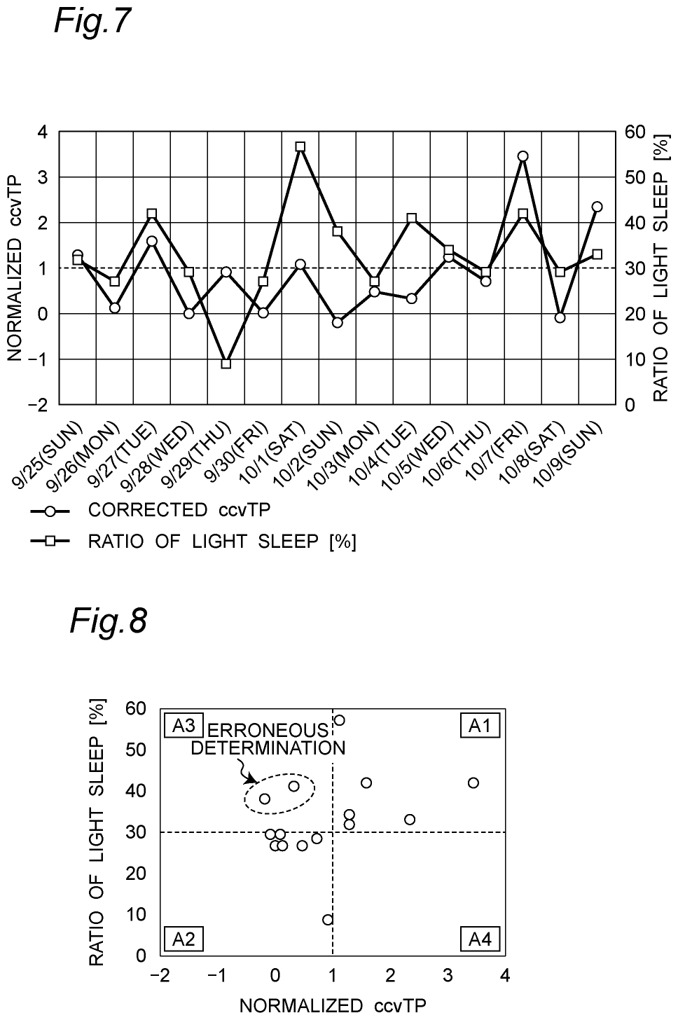

An example of the correlation between the autonomic nerve analysis result and the circadian rhythm change and the quality of sleep will be described with reference to FIGS. 7 to 11.

FIG. 7 shows an example of the correlation between the autonomic nerve analysis result, the circadian rhythm change, and the ratio of light sleep. FIG. 8 shows an example of determination of the ratio of light sleep based on the autonomic nerve analysis result and the circadian rhythm change. In FIGS. 7 and 8, ccvTP is used as the autonomic nerve analysis result. ccvTP represents the activity amount of the autonomic nerve. In general, the numerical value of ccvTP of a healthy young person is displayed high, and gradually decreases with aging. In addition, the numerical value of ccvTP is high in a healthy person, and is low in a person who suffers from fatigue and stress.

In FIGS. 7 and 8, ccvTP is weighted by the weighting coefficient K, and then normalized such that an average value becomes 0 and a threshold becomes 1. Hereinafter, the ccvTP weighted by the weighting coefficient K and then normalized is referred to as normalized ccvTP. Note that the weighting coefficient K is set to “1” within a period of −⅛ or more and ⅜ or less of the maximum value peak of the average circadian rhythm, and the other range is set to “0”.

The normalized ccvTP in FIGS. 7 and 8 means a maximum value within a period of −⅛ or more and ⅜ or less of the maximum value peak of the average circadian rhythm. When the value of the normalized ccvTP is equal to or more than a threshold of 1, it is determined that the normalized ccvTP is high. The threshold of the normalized ccvTP may be set to a different value depending on the user.

In the ratio of light sleep in FIGS. 7 and 8, the threshold is set to 30% according to the exemplary aspect. When the ratio of light sleep is equal to or less than the threshold 30%, it is determined that sleep is deep. The threshold of the ratio of light sleep may be set to a different value according to the user in alternative aspects.

As shown in FIG. 7, as the normalized ccvTP increases, the ratio of light sleep tends to increase. That is, as the normalized ccvTP increases, the sleep tends to be shallow. On the other hand, as the normalized ccvTP decreases, the ratio of light sleep tends to decrease. That is, as the normalized ccvTP decreases, the sleep tends to be deep. In FIG. 7, there is a portion different from the above-described tendency, but this is because an error is included.

As shown in FIG. 8, when the normalized ccvTP is equal to or more than the threshold of 1 and the ratio of light sleep is equal to or more than the threshold 30% (see the area A1 in FIG. 8), it can be determined that the normalized ccvTP is high and the sleep is light. Furthermore, when the normalized ccvTP is less than the threshold 1 and the ratio of light sleep is less than the threshold 30% (see the area A2 in FIG. 8), it can be determined that the normalized ccvTP is low and the sleep is deep. In FIG. 8, the areas A3 and A4 other than the areas A1 and A2 are erroneously determined.

Thus, there is a correlation between the normalized ccvTP and the quality of sleep. Thus, based on the correlation between the normalized ccvTP and the quality of sleep, the quality of sleep can be determined from the normalized ccvTP.

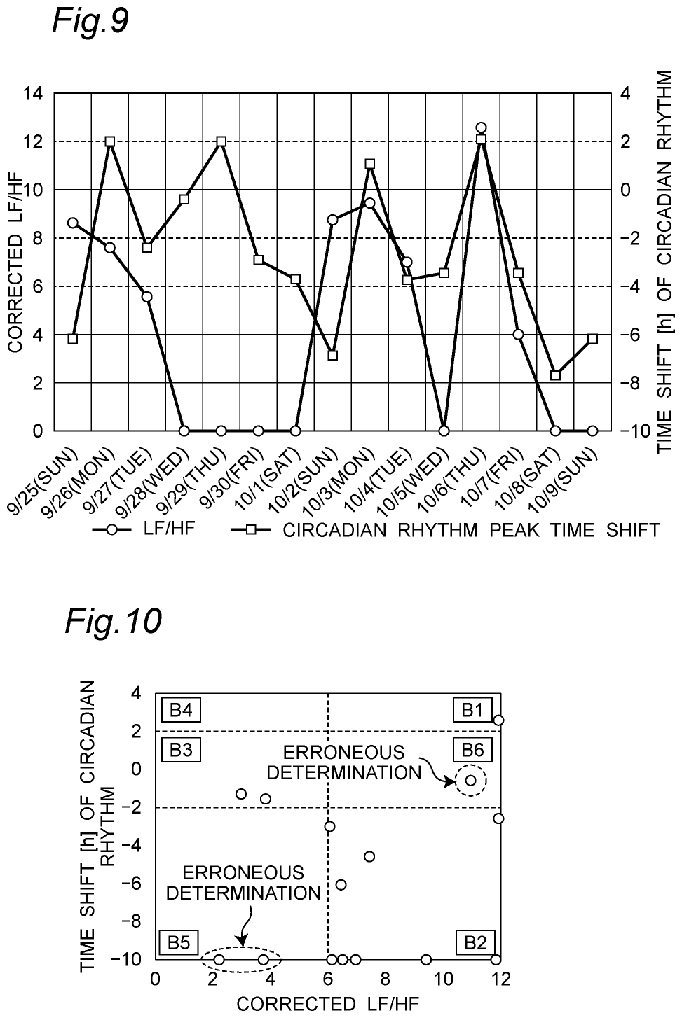

FIG. 9 shows an example of the correlation between the autonomic nerve analysis result and the time shift of the circadian rhythm. FIG. 10 shows an example of the determination of the time shift between the autonomic nerve analysis result and the circadian rhythm. In FIGS. 9 and 10, LF/HF is used as the autonomic nerve analysis result.

In FIGS. 9 and 10, LF/HF is corrected LF/HF weighted by the weighting coefficient K. It is noted that the weighting coefficient K is set to “1” within a period of −⅛ or more and ⅜ or less of the maximum value peak of the average circadian rhythm, and the other range is set to “0”.

In FIGS. 9 and 10, the corrected LF/HF means a maximum value within a period of −⅛ or more and ⅜ or less of the maximum value peak of the average circadian rhythm. The threshold of the corrected LF/HF is set to 6. When the value of the corrected LF/HF is equal to or more than the threshold of 6, it is determined that the corrected LF/HF is high. The threshold of the corrected LF/HF may be set to a different value according to the user.

The time shift of the circadian rhythm in FIGS. 9 and 10 means a shift from the predicted circadian rhythm calculated based on the corrected LF/HF for the average circadian rhythm. The threshold of the time shift of the circadian rhythm is set to 2 h. When the time shift of the circadian rhythm is 2 h or more, it is determined that the time shift of the circadian rhythm is large. In addition, in a case where it is difficult to determine a peak due to a decrease in the amplitude or multimodality of the circadian rhythm, the time shift is uniformly set to −10 h. It is noted that the threshold of the time shift of the circadian rhythm may be set to a different value according to the user. The time shift of the circadian rhythm may be an average of the shifts between the minimum value peak time and the maximum value peak time immediately after the corrected LF/HF measurement and the peak times of the next day.

As shown in FIG. 9, as the corrected LF/HF increases, the time shift of the circadian rhythm tends to increase. That is, as the corrected LF/HF increases, the change in the circadian rhythm tends to increase. On the other hand, when the corrected LF/HF decreases, the time shift of the circadian rhythm tends to decrease. That is, as the corrected LF/HF increases, the change in the circadian rhythm tends to decrease. In FIG. 9 there is a portion different from the above-described tendency, but this is because an error is included.

As shown in FIG. 10, when the corrected LF/HF is equal to or more than the threshold of 6 and the time shift of the circadian rhythm is equal to or more than the threshold of 2 h (see the areas B1 and B2 in FIG. 10), it can be determined that the corrected LF/HF is high and the time shift of the circadian rhythm is large. In addition, when the corrected LF/HF is less than the threshold of 6 and the time shift of the circadian rhythm is less than the threshold of 2 h (see the area B3 in FIG. 10), it can be determined that the corrected LF/HF is low and the time shift of the circadian rhythm is small. Further, in FIG. 10, the areas B4 to B6 other than the areas B1 to B3 are erroneously determined.

As described above, there is a correlation between the corrected LF/HF and the time shift of the circadian rhythm. Therefore, based on the correlation between the corrected LF/HF and the time shift of the circadian rhythm, the time shift of the circadian rhythm can be determined based on the autonomic nerve analysis result of the corrected LF/HF.

### [Output Displayed by Analysis System]

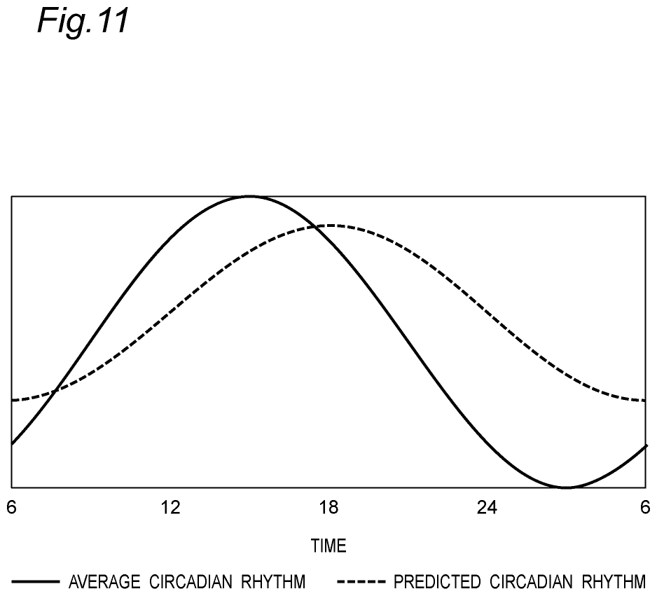

FIG. 11 shows an example of an output displayed by the analysis system according to the first exemplary embodiment. As shown in FIG. 11, the presentation unit 22 presents presentation information for improving the circadian rhythm created by the physical information analyzer 35. Specifically, the presentation information including the average circadian rhythm and the predicted circadian rhythm is presented on the presentation unit 22. As a result, the user can know the change (e.g., disturbance) of the circadian rhythm with respect to the average circadian rhythm by viewing the presentation information of the presentation unit 22.

### [Effects]

The analysis system 1A according to the first embodiment can achieve the following effects.

The analysis system 1A includes the biological data acquisition sensor 11, the circadian rhythm calculator 32, the autonomic nerve analyzer 33, the weighting coefficient calculator 34, and the physical information analyzer 35. The biological data acquisition sensor 11 acquires biological data and the circadian rhythm calculator 32 calculates the average circadian rhythm of the user. The autonomic nerve analyzer 33 performs the autonomic nerve analysis based on the variation of the biological data. The weighting coefficient calculator 34 calculates a weighting coefficient K for weighting the autonomic nerve analysis result analyzed by the autonomic nerve analyzer based on the measurement time at which the biological data of the user is measured and the cycle of the average circadian rhythm. The physical information analyzer 35 weights the autonomic nerve analysis result by the weighting coefficient K calculated by the weighting coefficient calculator 34, and estimates the change in the circadian rhythm with respect to the average circadian rhythm based on the weighted autonomic nerve analysis result. With such a configuration, a change in the circadian rhythm can be analyzed as one of the physical information. In addition, there is an advantage that the burden on the user when analyzing the physical information is small.

The circadian rhythm calculator 32 calculates the average circadian rhythm based on the biological data acquired by the biological data acquisition sensor 11. With such a configuration, the average circadian rhythm can be accurately calculated based on the biological data.

The analysis system 1A includes the input device 21 that inputs sleep information of the user. The circadian rhythm calculator 32 calculates the average circadian rhythm of the user based on the sleep information input by the input device 21. With such a configuration, the average circadian rhythm can be easily calculated based on the sleep information. For example, when the biological data is not sufficiently accumulated, the average circadian rhythm can be calculated based on the sleep information of the user. In a case where the information of the biological data is small, there is a possibility that an error is included in the calculation of the circadian rhythm. Therefore, for example, by calculating the average circadian rhythm based on the sleep information of the user until the biological data for one week or more is accumulated, it is possible to reduce an error when analyzing the physical information.

When the measurement time is in the range of the cycle of −⅛ or more and ⅜ or less of the maximum value peak of the average circadian rhythm, the weighting coefficient calculator 34 increases the weighting coefficient K as compared with the case where the measurement time is in the other range. With such a configuration, the physical information of the user can be analyzed with higher accuracy. By increasing the weighting of the autonomic nerve analysis result in the range of the cycle of −⅛ or more and ⅜ or less of the maximum value peak of the average circadian rhythm, the physical information can be analyzed with higher accuracy. The autonomic nerve analysis result in the range of the cycle of −⅛ or more and ⅜ or less of the maximum value peak of the circadian rhythm has a high correlation with the disturbance of the circadian rhythm. Therefore, by increasing the weighting coefficient in this range, the estimation accuracy of the change in the circadian rhythm can be improved.

The weighting coefficient K may be set for each user, or may be set according to the type of the autonomic nerve analysis result.

When the heart rate of the user is larger than a predetermined threshold, the physical information analyzer 35 corrects the weighted autonomic nerve analysis result. With such a configuration, it is possible to lower the reliability of the autonomic nerve analysis result based on the biological data acquired in a state where the sympathetic nerve is enhanced and the heart rate greatly increases, such as exercise or drinking that is not in a resting state. As a result, the physical information can be analyzed with higher accuracy. For example, a state in which the heart rate is temporarily higher than that in a normal state, such as during exercise or drinking, reduces the estimation accuracy of the circadian rhythm disturbance. By lowering the reliability of the autonomic nerve analysis result in the case of not being in such a resting state, the estimation accuracy of the change in the circadian rhythm can be improved.

The physical information analyzer 35 estimates a change in the circadian rhythm based on at least one of a time shift of a maximum value peak of the circadian rhythm from the average circadian rhythm, a time shift of a minimum value peak, a decrease in amplitude, and multimodality. As a result, the physical information can be analyzed with higher accuracy. When the circadian rhythm is disturbed, for example, there are a case where a shift of the maximum value peak time or a shift of the minimum value peak time occurs such as a time lag or shift work, and a case where the amplitude decreases such that the body temperature does not decrease at night. By differentiating and estimating them, the estimation accuracy of the influence on the quality of sleep and the like can be improved.

The physical information analyzer 35 estimates the quality of sleep and the activity preference of the user based on the weighted autonomic nerve analysis result. With such a configuration, more detailed physical information of the user can be analyzed.

The analysis system 1A includes the presentation unit 22 that presents presentation information including advice for improving the circadian rhythm. The physical information analyzer 35 creates the presentation information based on a change in the circadian rhythm. With such a configuration, the circadian rhythm of the user can be improved by presenting advice for improving the circadian rhythm to the user.

The physical information analyzer 35 calculates the predicted circadian rhythm based on the weighted autonomic nerve analysis result. The presentation information includes an average circadian rhythm and a predicted circadian rhythm. With such a configuration, the average circadian rhythm and the predicted circadian rhythm can be presented to the user, and notification of a change in the circadian rhythm can be provided.

The analysis system 1A includes the notification unit 22 that provides notification of the timing of measuring the biological data. With such a configuration, the user can know an appropriate timing to measure the biological data, and can measure the biological data in an appropriate state.

In the first embodiment, an example in which the analysis system 1A includes the measurement device 10, the control terminal 20, and the server 30 has been described, but the present invention is not limited thereto. In the analysis system 1A, these components may be realized by one device or may be realized by a plurality of devices. For example, the measurement device 10 and the control terminal 20 may be integrally formed. The measurement device 10, the control terminal 20, and the server 30 may be integrally formed. The measurement device 10 and the server 30 may be integrally formed.

It is noted that the components forming the analysis system 1A may be realized by a device other than the measurement device 10, the control terminal 20, and the server 30. For example, the components included in the measurement device 10, the control terminal 20, and the server 30 may be included in another device. As an example, the measurement device 10 may include the input device 21, the presentation unit 22, and/or the autonomic nerve analyzer 33. The control terminal 20 may include the biological data acquisition sensor 11, the circadian rhythm calculator 32, the autonomic nerve analyzer 33, and/or the weighting coefficient calculator 34. The server 30 may include the input device 21 and/or the presentation unit 22. Furthermore, the measurement device 10, the control terminal 20, and the server 30 may include elements other than the components shown in FIG. 1. Alternatively, the measurement device 10, the control terminal 20, and the server 30 may reduce the components shown in FIG. 1.

In the first embodiment, an example in which the analysis system 1A includes one measurement device 10 and one control terminal 20 has been described, but the present invention is not limited thereto. The analysis system 1A may include one or a plurality of measurement devices 10 and one or a plurality of control terminals 20. When the analysis system 1A includes the plurality of measurement devices 10 and/or the plurality of control terminals 20, information acquired by the plurality of measurement devices 10 and/or the plurality of control terminals 20 can be aggregated in the server 30. In the server 30, since the physical information can be analyzed using the information obtained from the plurality of users, the estimation accuracy of the physical information can be improved.

In the first exemplary embodiment, an example has been described in which the biological data includes the daily variation in vital information of at least one of the body temperature, the heart rate, the pulse rate, the respiration, the brain waves, and the blood pressure, but the present invention is not limited thereto. However, it should be appreciated that the biological data may include data other than these discussed above. For example, when the measurement device 10 includes the autonomic nerve analyzer 33, that is, when the measurement device 10 performs the autonomic nerve analysis based on the heart rate, the autonomic nerve activity index (LF, HF, LF/HF, TP, ccvTP) may be included as the biological data.

In the first embodiment, an example in which the analysis system 1A analyzes the physical information using the heart rate as the biological data has been described, but the present invention is not limited thereto. For example, the analysis system 1A may analyze the physical information using at least the heart rate or the pulse rate as the biological data. As a result, the biological data can be easily acquired, and the accuracy of the analysis of the physical information can be improved.

In the first embodiment, an example has been described in which the physical information analyzer 35 corrects the weighted autonomic nerve analysis result when the heart rate of the user is larger than the predetermined threshold, but the present invention is not limited thereto. The physical information analyzer 35 may correct the weighted autonomic nerve analysis result when the pulse rate of the user is larger than the predetermined threshold.

In the first embodiment, an exemplary analysis method has been described in which the components included in the measurement device 10, the control terminal 20, and the server 30 execute each step, but the analysis method is not limited thereto. It is noted that each step of the analysis method may be executed by a computer configured to perform the algorithms described herein. In particular, the exemplary computer includes a processor and a memory storing a program executed by the processor.

In the first embodiment, an example has been described in which the average circadian rhythm is calculated based on the sleep information and the biological data, but the present invention is not limited thereto. For example, the average circadian rhythm may be calculated based on the biological data without using the sleep information. In this case, the circadian rhythm calculator 32 may calculate the average circadian rhythm when the biological data for one week or more is accumulated. That is, the circadian rhythm calculator 32 may not calculate the average circadian rhythm until the biological data for one week or more is accumulated. Furthermore, the average circadian rhythm may be calculated based on the sleep information without using the biological data. Alternatively, the average circadian rhythm may be calculated based on information other than the sleep information. For example, the user may be caused to input information indicating whether the type is the morning type or the night type to the input device 21. The circadian rhythm calculator 32 may calculate the first circadian rhythm based on information of the type input by the user. The circadian rhythm calculator 32 only needs to be able to calculate the average circadian rhythm of the user, and can use arbitrary information.

In the first embodiment, the exemplary analysis method includes steps ST21 to ST29, but the analysis method is not limited thereto. As the steps of the analysis method, other steps may be added, some steps may be reduced, or a plurality of steps may be performed in one step as would be appreciated to one skilled in the art.

In the first embodiment, an example has been described in which the biological data acquisition sensor 11 includes the heart rate measurement device 14 and the body temperature measurement device 15, but the present invention is not limited thereto. The biological data acquisition sensor 11 may include a device configured for acquiring biological data. For example, the biological data acquisition sensor 11 may include a pulse rate measurement device, an activity amount measurement device, and the like.

In the first embodiment, an example has been described in which the biological data acquisition sensor 11 acquires the biological data when the user is awake, but the present invention is not limited thereto. For example, the biological data acquisition sensor 11 may acquire biological data of the user during sleep. As a result, the circadian rhythm calculator 32 can calculate the average circadian rhythm using the biological data of the user during sleep in addition to the biological data of the user when the user is awake. As a result, the average circadian rhythm more suitable for the user can be calculated.

In the first embodiment, an example in which the presentation unit 22 also functions as the notification unit has been described, but the present invention is not limited thereto. The presentation unit 22 and the notification unit may be separate components.

In the first embodiment, an example has been described in which the circadian rhythm calculator 32 calculates the average circadian rhythm using the variation in the body temperature of the user, but the present invention is not limited thereto. For example, the circadian rhythm calculator 32 may calculate the average circadian rhythm using the heart rate, the pulse rate, or the autonomic nerve activity index.

In the first embodiment, an example has been described in which the autonomic nerve analyzer 33 performs the autonomic nerve analysis based on the variation of the heart rate of the user, but the present invention is not limited thereto. For example, the autonomic nerve analyzer 33 may perform the autonomic nerve analysis based on the variation in the pulse rate of the user.

In the first embodiment, an example in which the time data is acquired by the measurement device 10 has been described, but the present invention is not limited thereto. For example, time data acquired by the control terminal 20 may be used as the time data. In this case, the control terminal 20 transmits a measurement start instruction to the measurement device 10 and receives the measurement data from the measurement device 10. At this time, the control terminal 20 may add time data and input information of the control terminal 20 to the measurement data and transmit the measurement data to the server 30.

### Second Exemplary Embodiment

An analysis system according to a second exemplary embodiment will be described. In the second embodiment, points different from the first embodiment will be mainly described. In the second embodiment, the same or equivalent configurations as those of the first embodiment will be described with the same reference numerals. In the second embodiment, the description overlapping with the first embodiment is omitted.

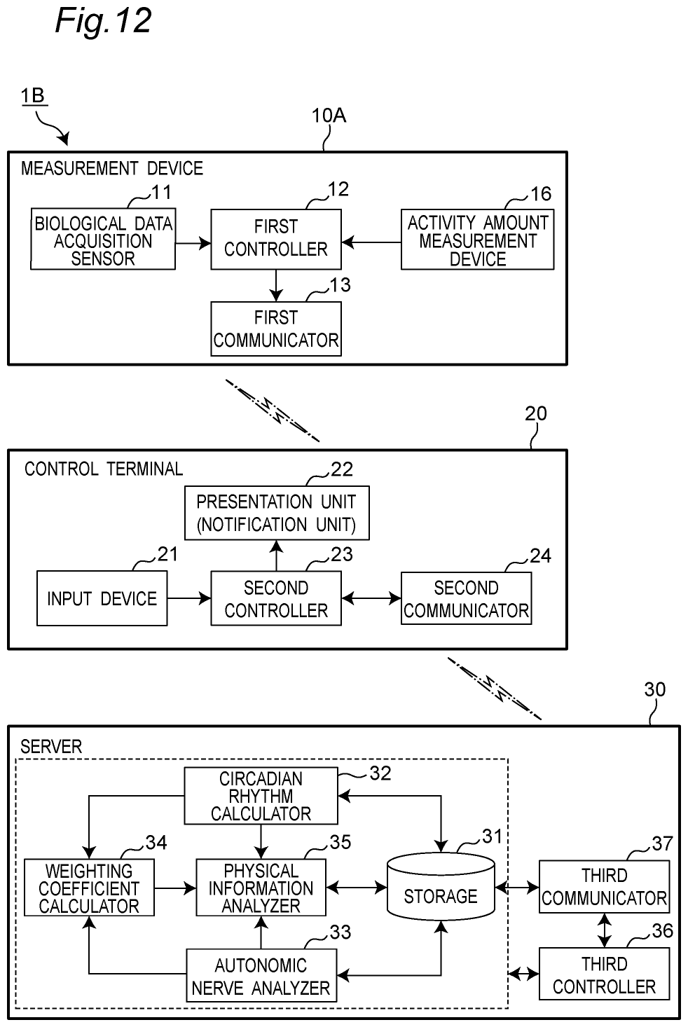

An example of the analysis system of the second embodiment will be described with reference to FIG. 12. FIG. 12 is a block diagram showing a schematic configuration of an example of an analysis system 1B according to the second embodiment.

The second embodiment is different from the first embodiment in that an activity amount measurement device 16 is provided. In particular, as shown in FIG. 12, the analysis system 1B further includes the activity amount measurement device 16.

### <Activity Amount Measurement Device>

The activity amount measurement device 16 is an activity meter that measures the activity amount of the user. The activity amount measurement device 16 is, for example, an acceleration sensor. The activity amount measurement device 16 is controlled by the first controller 12. The activity amount data of the user measured by the activity amount measurement device 16 is transmitted to the first controller 12. The first controller 12 transmits the activity amount data to the control terminal 20 via the first communicator 13. The control terminal 20 receives the activity amount data from the measurement device 10A by the second communicator 24, and transmits the activity amount data to the server 30.

In the second embodiment, since the analysis system 1B includes the activity amount measurement device 16, for example, the following processing can be realized.

### [Example of Calculation Processing of Average Circadian Rhythm Based on Activity Amount Data]

The circadian rhythm calculator 32 may calculate the first circadian rhythm based on the activity amount data. For example, the circadian rhythm calculator 32 estimates the bedtime and the wake-up time of the user based on the activity amount data, and calculates the first circadian rhythm based on the estimated bedtime and wake-up time of the user. For example, in a case where the activity amount data becomes smaller than a predetermined threshold and a state where the activity amount data is smaller than the predetermined threshold is continued for a predetermined time, the circadian rhythm calculator 32 determines that the user goes to bed and estimates the bedtime. On the other hand, when the activity amount data becomes larger than the predetermined threshold, the circadian rhythm calculator 32 determines that the user wakes up and estimates the wake-up time. When the activity meter is an acceleration sensor, the posture (e.g., decubitus, sitting/standing) can be determined from the acceleration information, so that the determination accuracy of going bed or waking up can be improved by combining the activity amount and the posture.

The circadian rhythm calculator 32 reads the correlation equation or the correlation table indicating the correlation between the bedtime, the wake-up time, and the circadian rhythm from the storage 31. The circadian rhythm calculator 32 calculates the first circadian rhythm using the estimated bedtime and wake-up time of the user and the read correlation equation or correlation table. The threshold of the activity amount data for estimating the bedtime may be different from or the same as the threshold of the activity amount data for estimating the wake-up time.

As described above, in the second embodiment, steps ST11 to ST13 shown in FIG. 4 of the first embodiment can be replaced with the arithmetic processing of the first circadian rhythm based on the activity amount data described above. Alternatively, the circadian rhythm calculator 32 can be configured to calculate the first circadian rhythm based on both the sleep information and the activity amount data.

With such a configuration, the accuracy of simple calculation of the first circadian rhythm can be improved. [Example of Determination Processing of Resting State Based on Activity Amount Data]

The measurement device 10A may determine whether or not the user is in a resting state based on the activity amount data, and acquire the biological data of the user by the biological data acquisition sensor 11 when the user is in the resting state. For example, the measurement device 10A determines that the user is not in the resting state when the activity amount data is larger than a predetermined threshold, and determines that the user is in the resting state when the activity amount data is equal to or smaller than the predetermined threshold. This determination is made by the first controller 12. In the measurement device 10A, the biological data acquisition sensor 11 acquires the biological data when the user is in the resting state.

When the activity amount data is equal to or more than the predetermined threshold, the measurement device 10A transmits information indicating that the user is not in the resting state to the control terminal 20 via the first communicator 13. The control terminal 20 creates presentation information for prompting the user to enter a resting state based on information indicating that the user is not in the resting state, and presents the presentation information to the presentation unit 22. Alternatively, when the activity amount data is smaller than the predetermined threshold, the measurement device 10A transmits information indicating that the user is in the resting state to the control terminal 20 via the first communicator 13. The control terminal 20 notifies the user of the timing of acquiring the biological data by the notification unit based on the information indicating that the user is in the resting state.

With such a configuration, the biological data can be acquired when the user is in the resting state. As a result, the accuracy of the autonomic nerve analysis is improved, and the estimation accuracy of the change in the circadian rhythm is improved. [Example of Autonomic Nerve Analysis Processing Based on Activity Amount Data]

The physical information analyzer 35 may correct the autonomic nerve analysis result weighted based on the activity amount data. For example, the physical information analyzer 35 acquires activity amount data via the control terminal 20. When the activity amount data is larger than the predetermined threshold, the physical information analyzer 35 decreases the correction coefficient K1. Alternatively, the physical information analyzer 35 may adjust the correction coefficient K1 based on the information of the heart rate data and the activity amount data.

With such a configuration, it is possible to determine whether or not the user is in the resting state based on the activity amount data. Furthermore, when the physical information analyzer 35 determines that the user is not in a stable state, the reliability of the autonomic nerve analysis result can be lowered. As a result, the accuracy of the autonomic nerve analysis result can be improved, and the estimation accuracy of the change in the circadian rhythm is improved.

When both the heart rate data and the activity amount data are used, whether the change in the heart rate is caused by exercise or the like or tension can be determined based on the activity amount data. As a result, the correction coefficient K1 can be changed according to the cause of increase in the heart rate, and the accuracy of the autonomic nerve analysis result can be improved.

All or part of the processing based on the activity amount data measured by the activity amount measurement device 16 described in the second embodiment may be performed.

In the second embodiment, an example has been described in which the activity amount measurement device 16 is included in the measurement device 10A, but the present invention is not limited thereto. For example, the activity amount measurement device 16 may be included in the control terminal 20.

In the second exemplary embodiment, an example has been described in which the measurement device 10A determines whether or not the user is in the resting state based on the activity amount data, but the present invention is not limited thereto. The control terminal 20 or the server 30 may determine whether or not the user is in the resting state based on the activity amount data.

An example in which the control terminal 20 includes the activity amount measurement device 16 and a global positioning system (GPS) will be described. In this case, the second controller 23 calculates the acceleration and the position of the user based on the activity amount data measured by the activity amount measurement device 16 and the GPS information, and calculates the exercise intensity and the movement history of the user. As a result, since the user's behavior can be analyzed, it is possible to save time and effort for the user to input information to the input device 21.

The control terminal 20 may control the measurement device 10 to start the measurement of the biological data by the user's input (for example, pressing the start button) to the input device 21. The measurement of the biological data is preferably performed when the user is in the resting state. Therefore, the control terminal 20 determines whether or not a large body motion has occurred during the measurement based on the activity amount data (e.g., acceleration data) measured by the activity amount measurement device 16. Then, in a case where it is determined that there is a large body motion, a warning alert or the like is presented from the presentation unit 22. In addition, in a case where there is a possibility that the accuracy of the calculation of the autonomic nerve activity index greatly decreases, the measurement may be automatically performed again.

Furthermore, instead of the user starting the measurement, the control terminal 20 may determine the resting state of the user from the activity amount data (e.g., acceleration data) and automatically start the measurement by the measurement device 10A. The control terminal 20 may determine that the user is in the resting state when the activity amount data (e.g., acceleration data) is not changed by a large amount for a predetermined time, and may automatically start the measurement by the measurement device 10A.

Furthermore, the control terminal 20 may constantly measure the activity amount data (e.g., acceleration data) to calculate the exercise intensity. The control terminal 20 may determine, from the exercise intensity, whether the user is walking, exercising, or resting before and after the measurement, and may determine the reliability of the analysis result. For example, in a case where the control terminal 20 determines that the user is exercising immediately therebefore, the reliability of measurement may be lowered. Since it takes time for the user to reach a resting state after exercise or walking, the control terminal 20 may not start measurement for a predetermined time. Furthermore, heart rate/pulse rate measurement may be performed at all times, a time zone in which the resting state continues for a time necessary for analysis during or after the measurement may be extracted, and analysis may be performed using data of the time zone.

### Third Exemplary Embodiment

An analysis system according to a third exemplary embodiment will be described. In the third embodiment, points different from the second embodiment will be mainly described. In the third embodiment, the same or equivalent configurations as those of the second embodiment will be described with the same reference numerals. In the third embodiment, the description overlapping with the second embodiment is omitted.

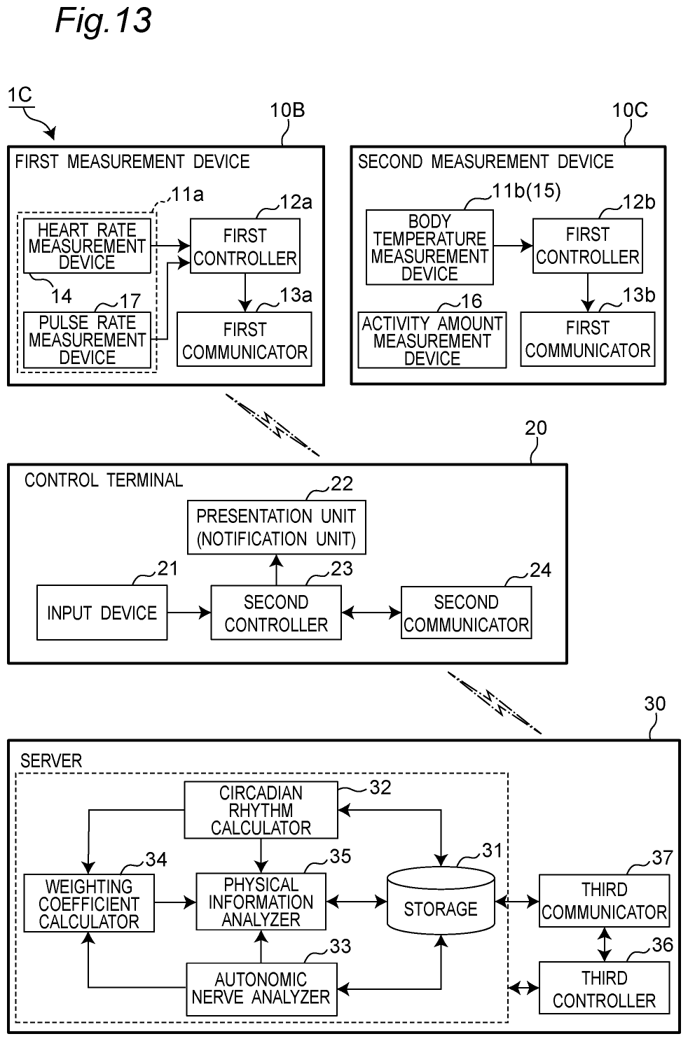

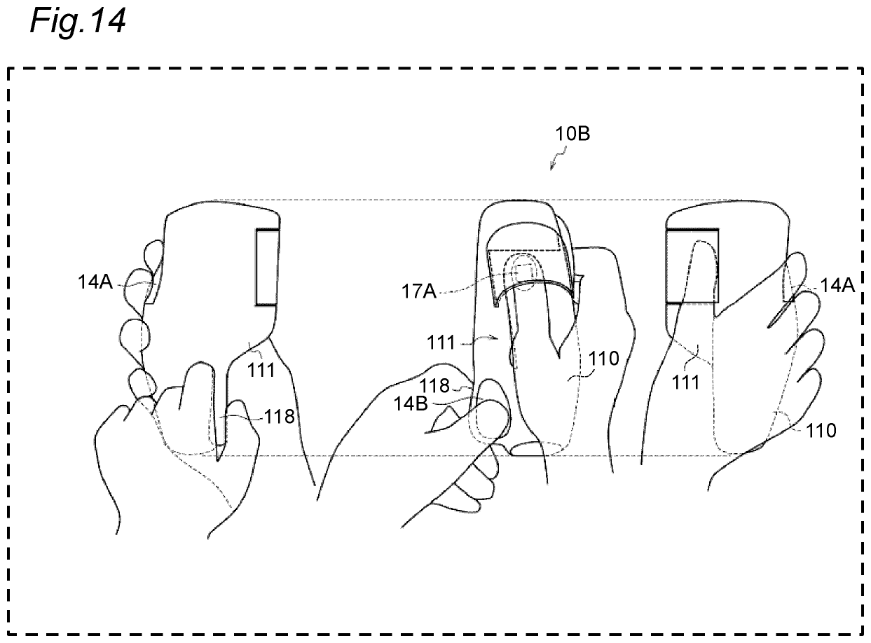

An example of an analysis system of the third embodiment will be described with reference to FIGS. 13 and 14. FIG. 13 is a block diagram showing a schematic configuration of an example of an analysis system 1C according to the third embodiment of the present invention. FIG. 14 is a schematic view of an example of a gripping-type measurement device.

The third embodiment is different from the second embodiment in that a first measurement device 10B and a second measurement device 10C are provided. In the third embodiment, the first measurement device 10B is a gripping-type device.

As shown in FIG. 13, the analysis system 1C includes the first measurement device 10B and the second measurement device 10C.

### <First Measurement Device>

The first measurement device 10B includes a biological data acquisition sensor 11a, a first controller 12a, and a first communicator 13a. The biological data acquisition sensor 11a includes the heart rate measurement device 14 and a pulse rate measurement device 17. In the first measurement device 10B, the heart rate data measured by the heart rate measurement device 14 and the pulse rate data measured by the pulse rate measurement device 17 are transmitted to the first controller 12a. The first controller 12a transmits the heart rate data and the pulse rate data to the control terminal 20 via the first communicator 13a.

### <Second Measurement Device>

The second measurement device 10C includes a biological data acquisition sensor 11b, the activity amount measurement device 16, a first controller 12b, and a first communicator 13b. The biological data acquisition sensor 11b includes the body temperature measurement device 15. In the second measurement device 10C, the body temperature data measured by the body temperature measurement device 15 and the activity amount data measured by the activity amount measurement device 16 are transmitted to the first controller 12b. The first controller 12b transmits the body temperature data and the activity amount data to the control terminal 20 via the first communicator 13b.

It is noted that the first controllers 12a and 12b and the first communicators 13a and 13b in the first measurement device 10B and the second measurement device 10C are similar to the first controller 12 and the first communicator 13 of the first embodiment, and thus detailed description thereof is omitted.

As shown in FIG. 14, the first measurement device 10B is a gripping-type measurement device. In the first measurement device 10B, the biological data acquisition sensor 11a that detects the heart rate and the pulse rate attaches electrocardiographic sensors (electrocardiographic electrodes) 14A and 14B and a photoelectric pulse wave sensor 17A to a portable gripping-type casing.

The first measurement device 10B is a gripping-type measurement device capable of acquiring an electrocardiographic signal and a photoelectric pulse wave signal by being gripped by the user and measuring a heart rate and a pulse rate body temperature. The first measurement device 10B has a main body 110 formed in a substantially spheroidal shape held by the user with the thumb and the other four fingers of one hand (for example, the right hand) at the time of measurement. On the side surface of the main body 110, a plate-shaped flange portion 118 is protruded in a direction substantially orthogonal to the protruding direction of the stopper portion 111 (that is, laterally). The flange portion 118 is provided to extend along the axial direction of the main body 110 (that is, from the proximal end portion side to the distal end portion side).

The first electrocardiographic electrode 14A is disposed such that a finger (for example, the index finger and/or the middle finger) of one hand (for example, the right hand) comes into contact with the first electrocardiographic electrode 14A when the main body 110 is gripped by the one hand. The first electrocardiographic electrode 14A may be disposed so as to be in contact with the thumb of one hand (for example, the right hand).

On the other hand, the second electrocardiographic electrode 14B formed in, for example, an elliptical shape for detecting an electrocardiographic signal is disposed on a front surface (and/or a back surface) of the flange portion 118. That is, the second electrocardiographic electrode 14B is disposed so as to be in contact with the finger (for example, the thumb and the index finger) of the other hand (for example, the left hand) by pinching (clamping) the flange portion 118 with the finger (for example, the thumb and/or the index finger) of the other hand. That is, when the user grips the main body 110 and the flange portion 118 of the first measurement device 10B, the left and right hands (e.g., fingertips) of the user come into contact with the first electrocardiographic electrode 14A and the second electrocardiographic electrode 14B, thereby acquiring an electrocardiographic signal corresponding to the potential difference between the left and right hands of the user.

The main body 110 is provided with the photoelectric pulse wave sensor 17A. The photoelectric pulse wave sensor 17A includes a light emitting element and a light receiving element, and acquires a photoelectric pulse wave signal from the fingertip of the thumb restricted by the stopper portion 111. The photoelectric pulse wave sensor 17A is a sensor that optically detects a photoelectric pulse wave signal using an absorption characteristic of blood hemoglobin.

As described above, the analysis system 1C may include the plurality of measurement devices 10B and 10C. In addition, the first measurement device 10B may be configured by a gripping-type device. That is, the biological data acquisition sensor 11a (the heart rate measurement device 14 and the pulse rate measurement device 17) may be attached to a gripping-type measurement device. As a result, the heart rate and the pulse rate can be easily measured.

In the third embodiment, an example in which the first measurement device 10B is a gripping-type device has been described, but the present invention is not limited thereto. For example, the second measurement device 10C may be a gripping-type device.

In the third embodiment, an example has been described in which the biological data acquisition sensor 11a includes the heart rate measurement device 14 and the pulse rate measurement device 17, but the present invention is not limited thereto. For example, the biological data acquisition sensor 11a may include either the heart rate measurement device 14 or the pulse rate measurement device 17. Alternatively, the biological data acquisition sensor 11a may include the body temperature measurement device 15.

In the third embodiment, an example has been described in which the second measurement device 10C includes the activity amount measurement device 16, but the present invention is not limited thereto. For example, the first measurement device 10B may include the activity amount measurement device 16, and the control terminal 20 may include the activity amount measurement device 16.

### Fourth Exemplary Embodiment

An analysis system according to a fourth exemplary embodiment will be described. In the fourth embodiment, points different from the second embodiment will be mainly described. In the fourth embodiment, the same or equivalent configurations as those of the second embodiment will be described with the same reference numerals. In the fourth embodiment, the description overlapping with the second embodiment is omitted.

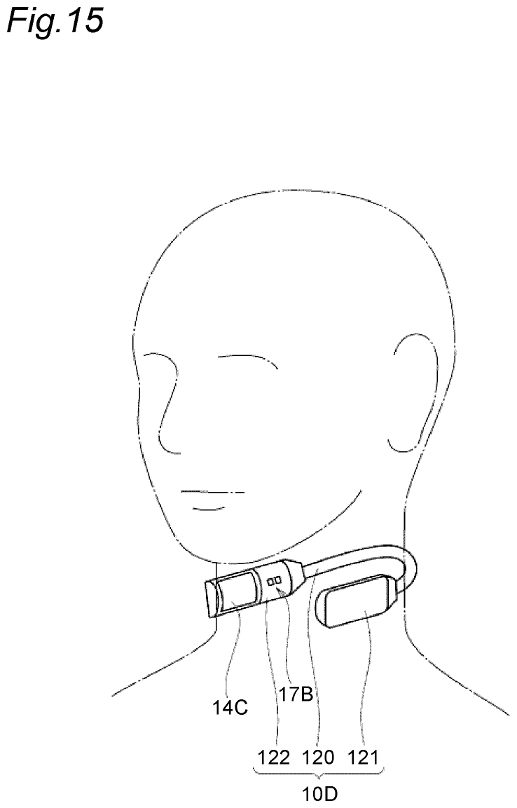

In the fourth embodiment, an example in which the measurement device is a wearing-type device or an attaching-type device will be described with reference to FIGS. 15 to 17. It is noted that the configuration of the analysis system of the fourth embodiment is similar to the configuration of the analysis system 1B of the second embodiment shown in FIG. 12, and thus detailed description is omitted.

### <Neck-Wearing-Type Device>

FIG. 15 is a schematic view of an example of a neck-wearing-type measurement device 10D. As shown in FIG. 15, the measurement device 10D includes a substantially U-shaped neck band 120 that is elastically worn so as to sandwich the neck from the back side of the neck of the user, and a pair of sensor units 121 and 122 that are disposed at both ends of the neck band 120 and are in contact with both sides of the neck of the user. The sensor unit 122 (121) mainly includes an electrocardiographic electrode (conductive cloth) 14C formed in a rectangular planar shape. Further, one sensor unit 122 includes a photoelectric pulse wave sensor 17B in addition to the above configuration. The photoelectric pulse wave sensor 17B optically detects a photoelectric pulse wave signal using an absorption characteristic of blood hemoglobin.

In this manner, the neck-wearing-type device is worn on the neck of the user. In the neck-wearing-type device, a configuration in which a pulse rate is measured by a photoelectric pulse wave sensor and a configuration in which a heart rate is measured by an electrocardiographic sensor including a plurality of electrocardiographic electrodes are both possible. The neck-wearing type has a relatively large sense of discomfort at the time of exercise or the like, but does not feel uncomfortable so much in daily life. In addition, the measurement stability may be second to that of the chest-attaching-type, and autonomic nerve activity measurement is sufficiently possible. In addition, the body surface temperature in the vicinity of the carotid artery is close to the core body temperature, and the core body temperature can be estimated similarly to the chest-attaching type, and the circadian rhythm can also be estimated from the core body temperature.

The measurement device 10D may include a temperature adjuster that adjusts the temperature of the neck of the user. For example, in a case where the decrease from the maximum value peak of the circadian rhythm does not occur, that is, in a case where the amplitude is small, by cooling the neck by the temperature adjuster, the body temperature of the user is lowered, and disturbance (e.g., a decrease in amplitude) of the circadian rhythm can be suppressed. In addition, if the core body temperature does not decrease at the time of falling asleep, the quality of sleep decreases. Therefore, the core body temperature can be decreased by cooling the neck by the temperature adjuster, and the falling asleep can be promoted. The temperature adjuster includes, for example, a Peltier element, a fan, and/or a blower as components to be cooled. This makes it possible to cool the neck by using a Peltier effect, blowing air to the neck, and heat of vaporization of water.

Furthermore, in a case where the increase from the minimum value peak of the circadian rhythm does not occur, that is, in a case where the amplitude is small, by heating the neck by the temperature adjuster, the body temperature of the user is raised, and disturbance (e.g., a decrease in amplitude) of the circadian rhythm can be suppressed. The temperature adjuster has, for example, a resistance, an infrared device, and/or a heater resistance as components to be heated. This configuration allows the neck to be warmed by emitting infrared radiation or directly heating the neck.

It is noted that the temperature adjuster may have at least one of a function of cooling and a function of heating according to various exemplary aspects.

### <Wristwatch-Type Device>

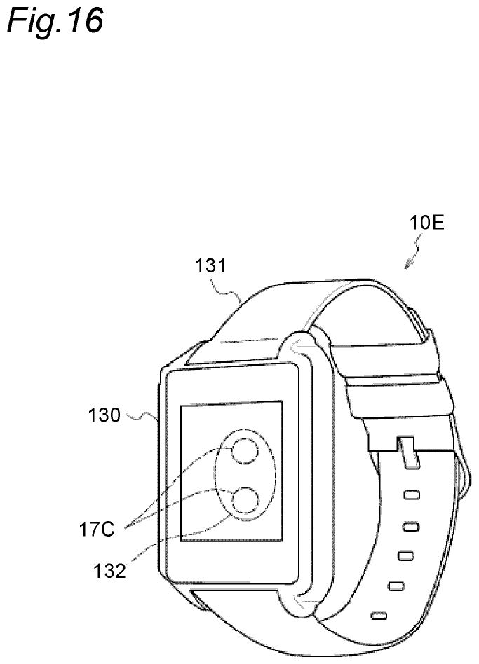

FIG. 16 is a schematic diagram of an example of a wristwatch-type measurement device 10E. As shown in FIG. 16, the wristwatch-type measurement device 10E includes a main body 130, a belt 131 attached to the main body 130, and a pulse wave sensor 132 disposed on the back surface of the main body 130. Moreover, a photoelectric pulse wave sensor 17C is disposed on the inner surface side of the pulse wave sensor 132. Therefore, when the user wears the wristwatch-type measurement device 10E on the wrist of one hand (for example, the left hand), the photoelectric pulse wave sensor 17C comes into contact with the wrist of the user, and the pulse wave number is measured.

### <Chest-Attaching-Type Device>

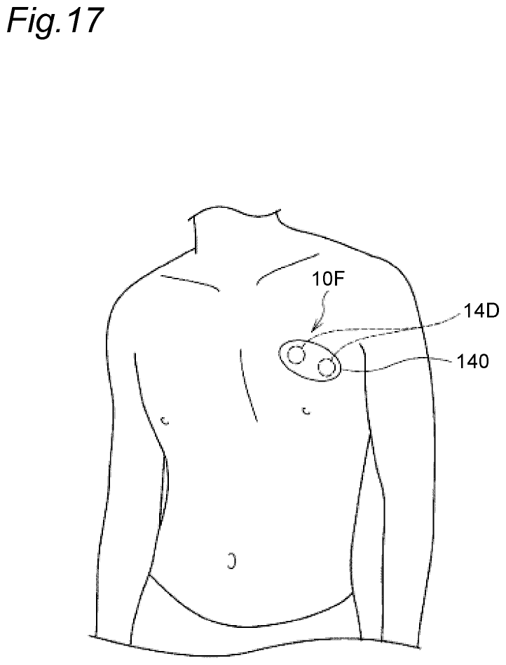

FIG. 17 is a schematic view of an example of a chest-attaching-type measurement device 10F. As shown in FIG. 17, the measurement device 10F includes a main body 140 that can be attached to the chest of the user, and two (or two or more) electrocardiographic electrodes (gel electrodes) 14D that are detachably attached to the main body 140. When an electrocardiographic signal or the like is measured using the measurement device 10F, the measurement device 10F is attached (worn) to the chest, and an electrocardiographic electrode (gel electrode) 14D is brought into contact with the chest. As a result, an electrocardiographic signal is detected by the electrocardiographic electrode (gel electrode) 14D.

As the electrocardiographic electrode 14D, for example, silver/silver chloride, a conductive gel, a conductive rubber, a conductive plastic, a metal, a conductive cloth, a capacitive coupling electrode in which a metal surface is coated with an insulating layer, or the like can be used. The metal is preferably, for example, stainless steel, Au, or the like which is resistant to corrosion and has less metal allergy. As the conductive cloth, for example, a woven fabric, a knitted fabric, or a nonwoven fabric made of a conductive yarn having conductivity is used. As the conductive yarn, for example, a resin yarn whose surface is plated with Ag or the like, a resin yarn coated with carbon nanotube coating, or a resin yarn coated with a conductive polymer such as PEDOT can be used. Further, a conductive polymer yarn having conductivity may be used.

The chest-attaching-type device preferably has a configuration in which a heart rate is measured by an electrocardiographic sensor having a plurality of electrocardiographic electrodes. The chest-attaching-type device has high measurement stability. In addition, since it is stuck to the trunk, the core body temperature (e.g., core temperature) can be estimated from the heat flux of the body surface temperature, and it is also possible to estimate the circadian rhythm from the core body temperature. In addition, it is also possible to fix to the chest with a belt instead of an adhesive tape.

In the fourth embodiment, an example in which the wearing-type device is worn on the neck and the arm has been described, but the present invention is not limited thereto. The wearing-type device may be worn on a portion other than the neck and the arm. For example, the wearing-type device may be worn on the chest. In addition, an example in which the attaching-type device is stuck to the chest has been described, but the present invention is not limited thereto. The attaching-type device may be stuck to a portion other than the chest. For example, the attaching-type device may be applied to the neck or arm. Even in such a configuration, the effects described in the wearing-type and attaching-type devices shown in FIGS. 15 to 17 can be achieved.

In the fourth embodiment, an example has been described in which the electrocardiogram electrodes 14C and 14D as the heart rate measurement device 14 and the photoelectric pulse wave sensors 17B and 17C as the pulse rate measurement device 17 are built in the wearing-type device and the attaching-type device, but the present invention is not limited thereto. For example, the body temperature measurement device 15 and/or the activity amount measurement device 16 may be built in the wearing-type device and the attaching-type device. As a result, the data of the body temperature and/or the activity amount can be easily acquired as the biological data of the user. In general, it is noted that although the embodiments of the present invention have been fully described in connection with exemplary embodiments with reference to the accompanying drawings, various modifications and corrections will be apparent to those skilled in the art.

1. A system for analyzing physical information and acquiring biological data of a user from a biological data acquisition sensor, the system comprising:
a circadian rhythm calculator configured to calculate an average circadian rhythm of the user based on the biological data acquired by the biological data acquisition sensor;
an autonomic nerve analyzer configured to perform an autonomic nerve analysis based on a variation in the biological data; and
a physical information analyzer configured to weight an autonomic nerve analysis result analyzed by the autonomic nerve analyzer with a weighting coefficient set based on a measurement time at which the biological data of the user is acquired and a cycle of the average circadian rhythm, and further configured to estimate a change in a circadian rhythm with respect to the average circadian rhythm based on the weighted autonomic nerve analysis result.

2. The system according to claim 1, further comprising a weighting coefficient calculator configured to set the weighting coefficient based on the measurement time at which the biological data of the user is acquired and the cycle of the average circadian rhythm.

3. The system according to claim 1, wherein the biological data includes at least a heart rate or a pulse rate.

4. The system according to claim 1, further comprising an input device configured to input sleep information of the user.

5. The system according to claim 4, wherein the circadian rhythm calculator is further configured to calculate the average circadian rhythm based on the sleep information input by the input device.

6. The system according to claim 1, wherein, when the measurement time is in a range of a cycle of −⅛ or more and ⅜ or less of a maximum value peak of the average circadian rhythm, the weighting coefficient calculator is configured to increase the weighting coefficient.

7. The system according to claim 1, wherein the physical information analyzer is further configured to correct the weighted autonomic nerve analysis result when a heart rate or a pulse rate of the user is larger than a predetermined threshold.

8. The system according to claim 1, wherein the physical information analyzer is further configured to estimate a change in the circadian rhythm based on at least one of a time shift of a maximum value peak of the circadian rhythm from the average circadian rhythm, a time shift of a minimum value peak, a decrease in amplitude, and multimodality.

9. The system according to claim 1, wherein the physical information analyzer is further configured to estimate a quality of sleep and activity preference of the user based on the weighted autonomic nerve analysis result.

10. The system according to claim 1, further comprising a presentation unit configured to present presentation information that includes advice for improving the circadian rhythm, with the physical information analyzer generating the presentation information based on a change in the circadian rhythm.

11. The system according to claim 10, wherein:
the physical information analyzer is further configured to calculate a predicted circadian rhythm based on the weighted autonomic nerve analysis result, and
the presentation information includes the average circadian rhythm and the predicted circadian rhythm.

12. The system according to claim 1, further comprising a notification unit configured to provide a notification of a timing at which the biological data is measured.

13. The system according to claim 1, wherein the biological data acquisition sensor is disposed in an attaching-type or wearing-type measurement device.

14. The system according to claim 13, wherein the measurement device is configured to be attached to or worn on a neck of the user, and includes a temperature adjuster configured to adjust a temperature of the neck of the user.

15. The system according to claim 1, further comprising an activity amount measurement device configured to measure activity amount data of the user, with the physical information analyzer correcting the weighted autonomic nerve analysis result based on the measured activity amount data.

16. The system according to claim 15, wherein the activity amount measurement device is attached to an attaching-type or a wearing-type measurement device.

17. A system for analyzing physical information and acquiring biological data of a user from at least one measurement device that includes a biological data acquisition sensor configured to acquire the biological data, the system comprising:
at least one control terminal configured to communicate with the at least one measurement device; and
a server configured to communicate with the at least one control terminal,
wherein the at least one of control terminal includes:
a presentation unit configured to present presentation information for improving a circadian rhythm; and
a communicator configured to transmit the biological data to the server and receive the presentation information from the server, and
wherein the server includes a circadian rhythm calculator configured to calculate an average circadian rhythm of the user based on the biological data acquired by the biological data acquisition sensor;
wherein the system further comprises:
an autonomic nerve analyzer configured to perform an autonomic nerve analysis based on a variation in the biological data of the user;
a physical information analyzer configured to weight an autonomic nerve analysis result analyzed by the autonomic nerve analyzer with a weighting coefficient set based on a measurement time at which the biological data of the user is acquired and a cycle of the average circadian rhythm, estimate a change in the circadian rhythm with respect to the average circadian rhythm based on the weighted autonomic nerve analysis result, and create the presentation information based on the change in the circadian rhythm; and
an additional communicator configured to receive the biological data from the control terminal and to transmit the presentation information to the control terminal.

18. The system according to claim 17, further comprising a weighting coefficient calculator configured to set the weighting coefficient based on a measurement time at which the biological data of the user is acquired and a cycle of the average circadian rhythm.

19. A computerized method for analyzing physical information comprising:
acquiring, by a biological data acquisition sensor, biological data of a user;
performing autonomic nerve analysis based on a variation in the biological data of the user;
calculating an average circadian rhythm of the user based on the biological data acquired by the biological data acquisition sensor;
weighting an autonomic nerve analysis result with a weighting coefficient set based on a measurement time at which the biological data of the user is acquired and a cycle of the average circadian rhythm; and
estimating a change in the circadian rhythm with respect to the average circadian rhythm based on the weighted autonomic nerve analysis result.

20. The method according to claim 19, further comprising setting the weighting coefficient based on the measurement time at which the biological data of the user is acquired and the cycle of the average circadian rhythm.
